(19) United States
(12) Patent Application Publication
(10) Pub. No.: US 2022/0370670 A1
(43) Pub. Date: Nov. 24, 2022

(54) DISINFECTING SYSTEMS, METHODS, AND DEVICES

(71) Applicant: MPICS INNOVATIONS PTE. LTD, Singapore (SG)

(72) Inventors: Kok Keong Richard LUM, Singapore (SG); Hong Sia TAN, Singapore (SG)

(21) Appl. No.: 17/328,665

(22) Filed: May 24, 2021

(51) Int. Cl. A61L 2/24; G06T 7/70; G06T 7/00; A61L 2/10; G06N 3/04; G06N 3/08; G01J 1/42

(52) U.S. Cl. CPC A61L 2/24; G06T 7/70; G06T 7/0002; A61L 2/10; G06N 3/04; G06N 3/08; G01J 1/429; G06T 2207/30244; G06T 2207/20081; H04W 56/001

(57) ABSTRACT

Disinfecting systems, methods, and devices are described. An example of a disinfecting system is disclosed to include: a radiation source configured to emit a disinfecting radiation; a source wireless communication device coupled to the radiation source; a plurality of receiver wireless communication devices; and a plurality of disinfecting radiation sensors configured to detect the disinfecting radiation, wherein each of the plurality of disinfecting radiation sensors is configured to record an intensity level information of the disinfecting radiation and configured to transmit the intensity level information through one of the plurality of receiver wireless communication devices to a center controller.

### FIELD OF TECHNOLOGY

The following relates to disinfection, including the disinfection of pathogens using ultraviolet (UV) light and UV sensors.

### BACKGROUND

Some cleaning techniques utilize ultraviolet (UV) radiation to remove bacteria and viruses from a physical environment, for example, from surfaces in the physical environment. In some cases, the amount of UV radiation energy a surface is exposed to during a disinfecting procedure may contribute to whether the surface is fully sanitized. For example, factors such as the radiant power of UV light applied during a disinfecting procedure, along with the duration over which the UV light is applied may determine whether the surface has been fully sanitized. Disinfection techniques are desired which may achieve a target effectiveness while minimizing related overhead (e.g., time, power, etc.).

### SUMMARY

The described techniques relate to improved methods, systems, devices, and apparatuses that support an ultraviolet (UV) disinfection system with sensors and feedback. The described techniques further relate to improved methods, systems, devices, and apparatuses that support a sensor device for detecting a disinfecting a state.

In one aspect, a system is provided that includes: a radiation source configured to emit UV radiation; one or more sensor devices, the one or more sensor devices including a sensor configured to detect the UV radiation; and a central controller. In some aspects, the one or more sensor devices are configured to record intensity information associated with the UV radiation. In some examples, the central controller may be configured to: aggregate the intensity information recorded by the one or more sensor devices; and evaluate one or more parameters associated with the radiation source based on the aggregated intensity information. In some examples, the system may include an image sensor configured to capture one or more images of a physical environment including the one or more sensor devices. In an example, evaluating the one or more parameters associated with the radiation source may be based on capturing the one or more images.

In some aspects, the central controller may be further configured to determine, based on the one or more images, at least one of: location information associated with the one or more sensor devices; orientation information associated with the one or more sensor devices; velocity information associated with the one or more sensor devices; and identification information associated with the one or more sensor devices.

In some aspects, the radiation source may be configured to generate a directed beam for communicating RF signals with the one or more sensor devices based on the location information, the orientation information, the velocity information, or a combination thereof; and the central controller may be further configured to establish a communications link with the one or more sensor devices based on the directed beam.

The system may include a machine learning network. In some aspects, the central controller may be further configured to: provide at least a portion of the aggregated intensity information, at least a portion of data associated with the one or more images, or both to a machine learning network; and receive an output from the machine learning network in response to the machine learning network processing at least the portion of the aggregated intensity information, at least the portion of the data, or both. In some aspects, evaluating the one or more parameters associated with the radiation source may be based on the output from the machine learning network.

In some aspects, the output from the machine learning network may include at least one of: a predicted radiation coverage corresponding to a target area and a temporal period; probability information corresponding to the predicted radiation coverage; and confidence information associated with the probability information. In some examples, the output from the machine learning network may include at least one of: predicted location information associated with the one or more sensor devices and a predicted temporal period; predicted orientation information associated with the one or more sensor devices and the predicted temporal period; and predicted velocity information associated with the one or more sensor devices and the predicted temporal period. In some other examples, the output from the machine learning network further may include at least one of: probability information corresponding to the predicted location information, the predicted orientation information, the predicted velocity information, or a combination thereof; and confidence information associated with the probability information.

In some aspects, based on the output from the machine learning network, the central controller may be further configured to control at least one of: a location of the radiation source; an emission direction of the radiation source; an emission power of the radiation source; and an emission duration of the radiation source.

In some aspects, the one or more sensor devices are configured to record temporal information associated with the intensity information recorded by the sensor, the temporal information including a timestamp value; and the central controller may be further configured to aggregate the intensity information based on the temporal information recorded by the one or more sensor devices.

In some aspects, the one or more sensor devices may include: a first sensor device configured to record first intensity information associated with the UV radiation, first temporal information associated with the first intensity information, or both; a second sensor device configured to record second intensity information associated with the UV radiation, second temporal information associated with the second intensity information, or both. In some aspects, the central controller may be configured to aggregate the first intensity information and the second intensity information based on comparison of the first temporal information and the second information. In some examples, a difference value between the first temporal information and the second information satisfies a threshold.

In some aspects, the one or more parameters associated with the radiation source may include at least one of: a degradation level associated with the emitted UV radiation; a degradation rate associated with the emitted UV radiation; a coverage area associated with the emitted UV radiation; and a quality level of a disinfection session.

In some aspects, the one or more sensor devices further may include: a housing; and a radiation sensitive material disposed on one or more portions of an external surface of the housing. In some examples, the image sensor may be further configured to visually detect the one or more sensor devices based on the one or more images, the radiation sensitive material, or both. In some examples, the radiation sensitive material may be disposed on the external surface of the housing according to a first pattern, the first pattern including a set of points formed of the radiation sensitive material; and the central controller may be configured to determine the location information, the orientation information, or both based on the set of points. In some aspects, the radiation sensitive material may be disposed on the external surface of the housing according to a second pattern, the second pattern including a one-dimensional pattern-based code formed of the radiation sensitive material, a multi-dimensional (e.g., two-dimensional) pattern-based code formed of the radiation sensitive material, or both; and the central controller may be configured to determine the identification information based on the one-dimensional pattern-based code, the multi-dimensional pattern-based code, or both. In some examples, the second pattern may be a one-dimensional pattern such as a barcode. In another example, the second pattern may be a two-dimensional version of the barcode, such as a Quick Response (QR) code.

In some aspects, the central controller may be configured to maintain a record of the intensity information recorded by the one or more sensor devices. In some aspects, the one or more sensor devices are configured to transmit the intensity information to the central controller. In some aspects, the central controller may be configured to maintain and update a record of location information associated with the one or more sensor devices. In some examples, the system may include a server; and a source device coupled to the radiation source. In some aspects, the central controller may be included in a processor of the server or the source device.

In some aspects, the one or more sensor devices are included in a target area of the system; and the one or more sensor devices are in an active state. In some aspects, the one or more sensor devices are configured to output a notification associated with the quality level of the disinfection session compared to a quality level threshold. In some aspects, the one or more sensor devices may include a calibration device configured to calibrate data measured by the sensor, the data including the intensity information associated with the UV radiation. In some aspects, the calibration device may include a dark current sensor.

In some aspects, the system may include: a first transceiver device configured for radio frequency (RF) communications; and a source device coupled to the first transceiver device. In some aspects, the source device may be configured to transmit, via the first transceiver device, temporal reference data. In some aspects, the one or more sensor devices are configured to synchronize with the temporal reference data.

In some aspects, the one or more sensor devices further may include: a second transceiver device configured for RF communications; and a wake-up device configured to generate a wake-up signal based on RF signals received at the second transceiver device. In some aspects, the one or more sensor devices are configured to enter an active state based on the wake-up signal. In some aspects, the one or more sensor devices may include a light sensor configurable to detect light having a wavelength between 380 nm to 780 nm. In some aspects, the one or more sensor devices are configured to enter an active state based on an amount of light detected by the light sensor satisfying a threshold.

In some aspects, the radiation source may include or may be electrically coupled to a location sensor: and the central controller may be further configured to determine, based on the location sensor of the radiation source, at least one of: location information associated with the radiation source; orientation information associated with the radiation source; and velocity information associated with the radiation source.

In some aspects, the one or more sensor devices may include a location sensor: and the central controller may be further configured to determine, based on the location sensor of the one or more sensor devices, at least one of: location information associated with the one or more sensor devices; orientation information associated with the one or more sensor devices; and velocity information associated with the one or more sensor devices.

In some aspects, the one or more sensor devices are configured to transmit an indicator based on at least one of: a comparison of the recorded intensity information to a first threshold; and a comparison of a pathogen level detected by the one or more sensor devices with respect to a target area to a second threshold. In some examples, the central controller may be further configured to pause emission of the UV radiation by the radiation source or resume the emission based on the indicator. In some examples, the central controller may be further configured to modify at least one of a position, a location, an orientation, and an emission direction of the radiation source based on the indicator.

In some aspects, the one or more sensor devices are configured to output a first notification associated with the comparison of the recorded intensity information and a threshold; the central controller may be configured to output a second notification associated with the comparison of the recorded intensity information and a threshold; or both. In some examples, the first notification, the second notification, or both may include at least one of: a visual notification; an audible notification; and a haptic notification.

In another aspect, a method at a system is provided that includes: emitting UV radiation via a radiation source; detecting the UV radiation; recording intensity information associated with the UV radiation; and aggregating the intensity information based on the recording. In some aspects, the method may include capturing, by an image sensor, one or more images of a physical environment including one or more sensor devices. In an example, the method may include evaluating the one or more parameters associated with the radiation source based on the aggregated intensity information, capturing the one or more images, or both. Based on the one or more images, the method may include determining at least one of: location information associated with the one or more sensor devices; orientation information associated with the one or more sensor devices; velocity information associated with the one or more sensor devices; and identification information associated with the one or more sensor devices.

In some aspects, the method may include generating a directed beam for communicating RF signals with the one or more sensor devices based on the location information, the orientation information, the velocity information, or a combination thereof; and establishing a communications link with the one or more sensor devices based on the directed beam.

In some examples, the method may include: providing at least a portion of the aggregated intensity information, at least a portion of data associated with the one or more images, or both to a machine learning network; and receiving an output from the machine learning network in response to the machine learning network processing at least the portion of the aggregated intensity information, at least the portion of the data, or both. In an example, evaluating the one or more parameters associated with the radiation source may be based on the output from the machine learning network.

The output from the machine learning network may include at least one of: a predicted radiation coverage corresponding to a target area and a temporal period; probability information corresponding to the predicted radiation coverage; and confidence information associated with the probability information. In some aspects, the output from the machine learning network may include at least one of: predicted location information associated with the one or more sensor devices and a predicted temporal period; predicted orientation information associated with the one or more sensor devices and the predicted temporal period; and predicted velocity information associated with the one or more sensor devices and the predicted temporal period. In some aspects, the output from the machine learning network may include at least one of: predicted location information associated with the one or more sensor devices and a predicted temporal period; predicted orientation information associated with the one or more sensor devices and the predicted temporal period; and predicted velocity information associated with the one or more sensor devices and the predicted temporal period. In some aspects, the output from the machine learning network further may include at least one of: probability information corresponding to the predicted location information, the predicted orientation information, the predicted velocity information, or a combination thereof; and confidence information associated with the probability information.

In some examples, the method may include controlling, based on the output from the machine learning network, at least one of: a location of the radiation source; an emission direction of the radiation source; an emission power of the radiation source; and an emission duration of the radiation source.

In some aspects, the method may include recording the intensity information may include recording temporal information associated with the intensity information, the temporal information including a timestamp value; and aggregating the intensity information may be based on the temporal information.

In some aspects, recording the intensity information may include: recording first intensity information associated with the UV radiation, first temporal information associated with the first intensity information, or both; and recording second intensity information associated with the UV radiation, second temporal information associated with the second intensity information, or both. In an example, aggregating the intensity information may include aggregating the first intensity information and the second intensity information based on comparison of the first temporal information and the second information.

In some aspects, the one or more parameters associated with the radiation source may include at least one of: a degradation level associated with the emitted UV radiation; a degradation rate associated with the emitted UV radiation; a coverage area associated with the emitted UV radiation; and a quality level of a disinfection session.

In some aspects, the method may include receiving an indicator from one or more sensor devices included in a physical environment. In some aspects, the indicator may be associated with intensity information recorded by the one or more sensor devices, a pathogen level detected by the one or more sensor devices, or both. In some examples, the method may include pausing emission of the UV radiation by the radiation source or resuming the emission. based on the receiving the indicator. In some cases, the method may include modifying at least one of a position, a location, an orientation, and an emission direction of the radiation source based on the indicator.

In another aspect, a device is provided that includes: a housing; a radiation sensitive material disposed on one or more portions of an external surface of the housing; a sensor configured to measure intensity information associated with UV radiation of a first frequency band; a controller; and a transceiver configured to transmit and receive radio frequency (RF) signals. In some aspects, the controller may be configured to record the intensity information, temporal information associated with measuring the intensity information, or both. In some examples, the temporal information may include a timestamp value.

In some aspects, the device may include a calibration device configured to calibrate data measured by the sensor, the data including the intensity information associated with the UV radiation. In some examples, the calibration device may include a dark current sensor. In some aspects, the device may be configured to synchronize with temporal reference data.

The device may include a wake-up device configured to generate a wake-up signal based on RF signals received at the transceiver device. In some aspects, the device may be configured to enter an active state based on the wake-up signal.

The device may include a light sensor configurable to detect light having a wavelength between 380 nm to 780 nm. In some aspects, the device may be configured to enter an active state based on an amount of the light detected by the light sensor satisfying a threshold.

In some aspects, the radiation sensitive material may be disposed on the external surface of the housing according to a first pattern, a second pattern, or both. In some aspects, the first pattern may be asymmetrical with respect to one or more axes of the sensor device. In some aspects, the first pattern may be asymmetrical with respect to all axes of the sensor device. In some aspects, the first pattern may be asymmetrical in a two-dimensional plane. In some examples, the first pattern may include a set of points formed of the radiation sensitive material. In some other examples, the first pattern may include a polygon shape corresponding to the set of points, the polygon shape including a set of internal angles that are different from one another. In some aspects, the polygon shape may be a quadrilateral shape; and the set of internal angles may include four internal angles that are different from one another. In some examples, the second pattern may include a pattern-based code including identification information associated with the device. For example, the second pattern may include a one-dimensional pattern-based code, a multi-dimensional (e.g., two-dimensional) pattern-based code, or both. In some examples, the second pattern may be a one-dimensional pattern such as a barcode. In another example, the second pattern may be a two-dimensional version of the barcode, such as a QR code.

In some aspects, the radiation sensitive material has a first color when exposed to the UV radiation of the first frequency band; and the radiation sensitive material has a second color in an absence of the UV radiation of the first frequency band or when exposed to UV radiation of a second frequency band. In some aspects, the first frequency band does not overlap with the second frequency band.

The device may include a UV filter positioned at an opening of the housing. In some aspects, the UV filter may be configured to pass the UV radiation of the first frequency band. In some aspects, the sensor may include a photodetector positioned to receive the UV radiation passed by the UV filter. In some aspects, the sensor may be configured to detect the UV radiation of the first frequency band, and the first frequency band may be from 100 nm to 280 nm.

In some aspects, the device may include a location sensor configured to record at least one of location information, orientation information, and velocity information associated with the device.

In some aspects, the device may be configured to transmit an indicator based on at least one of: a comparison of the recorded intensity information and a first threshold; and a comparison of a pathogen level associated with a target area and a second threshold. In some aspects, the device may be configured to output a notification based on a comparison of the recorded intensity information and a first threshold. In some examples, the notification may include at least one of: a visual notification; an audible notification; and a haptic notification.

In another aspect, a method at a device is provided that includes: receiving temporal reference data; detecting UV radiation of a first frequency band; measuring intensity information associated with the UV radiation of the first frequency band; and recording the intensity information, temporal information associated with measuring the intensity information, or both. The temporal information may include a timestamp value synchronized with the temporal reference data. In some aspects, the method may include receiving RF signals during a temporal period; and entering an active state based on receiving the RF signals. In some examples, the first frequency band may be from 100 nm to 280 nm.

In some examples, the method may include transmitting an indicator based on at least one of: comparing the recorded intensity information and a first threshold; and comparing a pathogen level associated with a target area and a second threshold. In some aspects, the indicator may include a command associated with pausing or enabling emissions of the UV radiation. In some other examples, the method may include outputting a notification based on a comparison of the recorded intensity information and a first threshold. The notification may include at least one of: a visual notification; an audible notification; and a haptic notification.

In some examples, the method may include detecting, at a light sensor of the device, light having a wavelength between 380 nm to 780 nm; and entering an active state based on an amount of the light detected by the light sensor satisfying a threshold.

### BRIEF DESCRIPTION OF THE DRAWINGS

FIG. 1 illustrates an example of a system that supports an ultraviolet (UV) disinfection system with sensors and feedback in accordance with aspects of the present disclosure.

FIG. 2 illustrates an example of a sensor tag that supports a UV disinfection system with sensors and feedback in accordance with aspects of the present disclosure.

FIG. 3 illustrates an example of a sensor tag that supports a UV disinfection system with sensors and feedback in accordance with aspects of the present disclosure.

FIG. 4 illustrates example captured images of a sensor tag that supports a UV disinfection system with sensors and feedback in accordance with aspects of the present disclosure.

FIGS. 5A and 5B illustrate example configurations of a sensor tag that supports a UV disinfection system with sensors and feedback in accordance with aspects of the present disclosure.

FIG. 6 illustrates example captured images of a sensor tag that supports a UV disinfection system with sensors and feedback in accordance with aspects of the present disclosure.

FIG. 7 illustrates an example of a system that supports a UV disinfection system with sensors and feedback in accordance with aspects of the present disclosure.

FIG. 8 illustrates an example of a process flow of a system that supports a UV disinfection system with sensors and feedback in accordance with aspects of the present disclosure.

FIG. 9 illustrates an example of a process flow of a sensor tag that supports a UV disinfection system with sensors and feedback in accordance with aspects of the present disclosure.

### DETAILED DESCRIPTION

According to example aspects of the present disclosure, an ultraviolet (UV) disinfection system is provided which includes a radiation source and a source device (also referred to herein as a source wireless communication device) coupled to the radiation source. In some aspects, the radiation source may be a UV light source configured to emit light in the UV range. For example, the radiation source may be configured to emit light having a wavelength between 200 nm to 300 nm (i.e., UV-C wavelength range).

The UV disinfection system may include a series of sensor tags (also referred to herein as UV tags, UV sensor tags, or sensor devices). The sensor tags may be, for example, communication devices capable of transmitting and receiving signals (e.g., via wired or wireless communications). In some aspects, each of the sensor tags may include one or more radiation sensors (also referred to herein as light sensors, UV sensors, or electronic UV sensors). In an example, a radiation sensor included in a sensor tag may detect and measure UV radiation (e.g., UV-C radiation).

In some aspects, a radiation sensor of a sensor tag may convert detected UV radiation into digital data (e.g., a digital measurement value), based on which the UV disinfection system may calculate the total UV radiation (e.g., total energy) emitted by the radiation source into the physical environment. In some examples, each sensor tag may communicate measured UV radiation power at the sensor tag (e.g., the total energy of UV radiation). For example, each sensor tag may communicate measured UV radiation power to other devices in the UV disinfection system using radio frequency (RF) communications (e.g., via an RF transceiver included in the sensor tag).

In some examples, a sensor tag may be powered by an electrical power source electrically coupled to the sensor tag. For example, the electrical power source may include an external power source. In another example, the electrical power source may include batteries integrated with the sensor tag (e.g., included in a housing of the sensor tag). The batteries may include long-life or extended-life batteries. In some aspects, the sensor tags (e.g., battery powered, or coupled to an electrical power source) may be attached to any area in a room or a target area. In some examples, the locations of the sensor tags may calculate in consideration of disinfecting the target area (e.g., calculated such that the readings of the sensor tags can be computed to deduce whether the target area is sufficiently disinfected). Sufficient disinfection, for example, may encompass a disinfection level or disinfection coverage (e.g., based on UV emission power, UV emission duration, UV emission coverage) high enough to kill known viruses or germs or meet some standards (e.g., threshold). Power consumption of such sensor tags, for example, may be designed (e.g., usually designed) to be low power consumption.

In some other aspects, the sensor tag may be powered by energy harvesting techniques including solar, piezo-vibrational, or wireless charging. In an example, the sensor tag may be powered by RF wireless waves (e.g., RF wireless charging). For example, the source device may include an RF transceiver, via which the source device may wirelessly power or charge the sensor tag. In some aspects, the UV disinfection system may energize sensor tags (within an RF range of the RF transceiver coupled to the source device) to provide power to enable UV sensing operations of the sensor tags. In some other aspects, the sensor tags may be energized by background RF signals (e.g., Bluetooth, WiFi, 3G, 4G, or 5G sources generated by other communications devices).

In some aspects, the RF transceiver of a sensor tag may include one or more tag-printed coil antennas. In an example, the sensor (e.g., UV sensor) included in the sensor tag may enter an awake state when an amount of RF energy received at the RF transceiver (e.g., extracted RF energy) is greater than or equal to a threshold. In some aspects, in the awake state, the sensor tag (and the included sensor) may operate in an ultra-low power mode. For example, the sensor tag may implement ultra-low power sensor operations including dark current mitigation and cancellation for the sensor (e.g., UV sensor) included in the sensor tag, data conversion (e.g., analog-to-digital (ADC) conversion) of UV intensity measured at the sensor, data calibration, and data storage.

The UV disinfection system may support a global wake-up of sensor tags (and sensors thereof) included in the UV disinfection system. The global wake-up feature may enable the sensor tags to remain in low power mode or in hibernation mode to reduce power consumption. The sensor tags included in the UV disinfection system may be, for example, sensor tags located within a RF coverage area of the UV disinfection system (e.g., based on RF transmission power, RF signal quality, a distance threshold associated with the RF transceiver of the source device). In some aspects, the UV disinfection system may support temporal synchronization among the sensor tags included in the UV disinfection system. For example, via the RF transceiver, the source device may transmit temporal reference data (e.g., time data) to all sensor tags included in the UV disinfection system. In an example, each sensor tag may report UV levels measured by an included sensor, in combination with temporal information (e.g., timestamp data) corresponding to the measurements.

In some aspects, the source device may read digital data generated by the sensor tag. The sensor tag may communicate the digital data to the source device, for example, over an established communications link (e.g., RF communications link). In an example, the digital data may include UV radiation information and time information. For example, the digital data may include measured UV radiation levels and timestamps corresponding to when the UV radiation levels are measured by a sensor tag and/or read by the source device. In some examples, the sensor tags may be assigned respective unique identifiers, and the UV disinfection system may reference the identifiers to distinguish between UV data respectively measured by different sensor tags.

The UV disinfection system may include an image sensor. In an example, the image sensor and the radiation source may be positioned or located within the UV disinfection system such that, a relative position between the image sensor and the radiation source is configured or known. Based on the relative positioning, the image sensor and the radiation source may share a same frame of reference (e.g., a shared reference point). In some aspects, the image sensor may capture images of a physical environment to be sanitized by the UV disinfection system, using one or more captured images (e.g., background images of the physical environment) as a reference for determining characteristics associated with the physical environment, characteristics of sensor tags within the physical environment (e.g., distance from the image sensor and/or the radiation source to the sensor tags), or characteristics of physical objects within the physical environment (e.g., distance from the image sensor and/or the radiation source to the physical objects). In some examples, the image sensor may detect, calculate, and record geolocation information (e.g., coordinates, global positioning satellite (GPS) coordinates) of the sensor tags with respect to the physical environment. The geolocation information may include a local map or a local relative positioning of the sensor tags within a building.

In some aspects, each sensor tag may include a set of markers. The markers may also be referred to herein as UV markers, visual markers, luminous markers, targets, or points. In some cases, a set of markers may be referred to as an object (e.g., a set of four markers may be referred to as a four-point object). Each of the markers may include a UV coating (e.g., a UV luminous paint). In some cases, the image sensor may identify and locate a sensor tag based on the markers. In one embodiment, the marker may comprise a line and the four markers may include a non-symmetrical quadrilateral shape. For example, the image sensor may distinguish sensor tags from other reference objects in a physical environment, based on the markers included in the sensor tags. For example, the image sensor may identify the markers based on a response (e.g., of the respective UV coatings) to UV light emitted by the radiation source.

According to example aspects of the present disclosure, the UV disinfection system may emit UV radiation directed toward a target surface. In an example, the system may identify sensor tags and respective sensor tag locations through visual means (e.g., based on images captured by the image sensor and/or detection of respective markers). For example, for each sensor tag, the UV disinfection system may support visual calculation and estimation of location information (e.g., coordinates, positioning) of the respective markers. The UV disinfection system may determine direction and distance of the markers (and corresponding sensor tags), for example, in relation to the radiation source and/or the image sensor.

Based on the location information of the markers (and corresponding sensor tags), the UV disinfection system may apply RF beamforming techniques for directing RF signals from the source device to the sensor tags. In some cases, based on the location information, the UV disinfection system may apply the RF beamforming techniques to establish a communications link (e.g., a directed RF link) between the source device and the sensor tags. In some aspects, the directed RF link may support improved RF energy transfer (e.g., higher efficiency) to the sensor tags. In some other aspects, the directed RF link may support improved RF data communications (e.g., increased RF communications quality, increased quality of service (QoS)) between the source device and the sensor tags.

The UV disinfection system may collect and aggregate data from the sensor tags, in combination with identification information associated with the sensor tags. The identification information may include, for example, unique identifiers (UIDs) associated with the sensor tags. In some examples, the data may include measured UV radiation levels (also referred to herein as intensity information) and corresponding temporal information. Based on aggregating the data, the UV disinfection system may map topological information (e.g., location information, orientation information) associated with the sensor tags.

In some aspects, based on the aggregated data, the UV disinfection system may determine the amount of UV light incident a physical environment and/or any target surface of the physical environment. For example, the UV disinfection system may determine the amount of surface radiation (e.g., UV radiation coverage) and time duration (e.g., radiation duration) with respect to the physical environment and/or target surfaces. In some aspects, the system may process the aggregated data using any combination of data analytics, machine learning and artificial intelligence (AI) processing. In some examples, the UV disinfection system may transfer the aggregated data or any portion thereof to cloud-based data storage or a server (e.g., a cloud-based server) via wired or wireless communication.

A machine learning network (e.g., implemented in the UV disinfection system, the source device, or the server) may evaluate the data and generate feedback information (e.g., probability information, confidence information) with respect to the UV radiation coverage associated with the physical environment and/or target surface. For example, the machine learning network may generate and output feedback information with respect to the amount of UV radiation energy detected by radiation sensors of a sensor tag. In some aspects, the feedback information may be associated with the total amount of UV radiation energy detected by radiation sensors of multiple sensor tags (e.g., all sensor tags included in the physical environment). In some cases, based on an evaluation of the data, the machine learning network may predict the amount of UV radiation energy incident one or more target surfaces in the physical environment (e.g., UV radiation coverage) in relation to a predicted amount of time.

The system may collect and provide UV data to the machine learning network periodically (e.g., based on a schedule, a temporal duration, etc.) or in real-time. In some examples, based on the feedback information provided by the machine learning network, the system may set or adjust one or more parameters of the radiation source. For example, the system may adjust the output power of the radiation source, reposition the radiation source (e.g., modify an emission direction of the radiation source), or modify a location of the radiation source based on the feedback information.

In some aspects, the system may aggregate location information of the sensor tags. The system (or the machine learning network) may analyze the location information, for example, to verify the operation of the sensor tags. For example, the system may identify whether any sensor tags have been tampered with, incorrectly located by the system (e.g., not detected), or damaged. For example, the identification may be accomplished by comparing existing or previously stored marker data (e.g., location information associated with markers of the sensor tags) to current marker data. In yet another example, the geolocation information may be utilized for the same purpose (e.g., aggregating location information, analyzing location information). In some aspects, the system may identify whether any sensor tags are non-visible to the radiation source. For example, the system may identify whether an object located between the radiation source and a sensor tag is preventing UV light emitted by the radiation source from reaching the sensor tag.

The UV disinfection system may support synchronization between the source device and the sensor tags (e.g., time synchronization) in combination with various system level functionality. In some aspects, the UV disinfection system may support time synchronization between any of the source device, the RF transceiver of the source device, the radiation source, the image sensor, and the sensor tags in combination with system level functionality. For example, the UV disinfection system may support control (e.g., via commands, signals, etc.) of any of the source device, the RF transceiver of the source device, the radiation source, the image sensor, and the sensor tags. In an example, the UV disinfection system may support a central controller implemented at the source device or at a server (e.g., a cloud-based server, a local server) of the UV disinfection system.

In an example, the UV disinfection system may support controlling the emission of UV radiation based on the data aggregated from the sensor tags (e.g., as measured by the sensors). For example, the UV disinfection system may position the radiation source and/or direct emissions of the radiation source based on the data. In another example, the UV disinfection system may position and/or direct the RF transceiver of the source device based on the data aggregated from the sensor tags and image data captured by the image sensor (e.g., visual confirmation information by the image sensor with respect to sensor tag locations).

A sensor tag may include multiple markers. The system may detect the markers of the sensor tag (e.g., using the image sensor), and based on the detected markers, the system may identify location information of the sensor tag (or a radiation sensor included therein). For example, the system may use the location information to estimate the distance between the image sensor and the sensor tag. In another example, the system may calculate a viewing angle and/or distance from the image sensor to the sensor tag. Based on the relative positioning between the image sensor and the radiation source, the system may calculate an angle and/or distance from the radiation source to the sensor tag.

In some aspects, the markers may be UV luminous markers. For example, the markers may be formed of a radiation sensitive material disposed on a housing of the sensor tag. In an example, the radiation sensitive material may react (e.g., change color) in response to light having a wavelength in the UV-C wavelength range. For example, the radiation sensitive material may transition between different colors based on an exposure to UV-C light emitted by the radiation source.

In some examples, a sensor tag may include a minimum of four markers. In an example, the markers may represent four respective points (e.g., X-axis and Y-axis coordinates) in the Cartesian plane. Using the four points in a captured image, for example, the system (or machine learning network) may calculate or estimate the distance between the image sensor (or the radiation source) and the sensor tag. In one embodiment, the sensor tag may include four linear lines, and intersection points (e.g., representative of the four respective points) may be detected and recognized by the image sensor. In another aspect, using different images captured at different temporal instances, the machine learning network may predict a distance between the image sensor (or the radiation source) and the sensor tag with respect to a predicted temporal period (e.g., a future temporal instance). In some aspects, the system may support adjusting the resolution of the image sensor and/or optical zoom settings to increase the accuracy with respect to calculated distance and angle.

In some aspects, the markers on a sensor tag may be asymmetric with respect to a two-dimensional (2D) plane. For example, the markers may be asymmetric with respect to any axis associated with the sensor tag. Based on the asymmetry, for example, the image sensor may avoid capturing the same reference image (e.g., having the same marker positions and/or marker sizes) when capturing images of a sensor tag from different locations or perspectives. In an example of aspects of the present disclosure, the markers may be positioned on a sensor tag such that the markers are not located along shapes having an axis of symmetry (e.g., an ellipse).

In an example of a four-point object without an axis of symmetry, a set of four markers may be positioned asymmetric to one another. The four markers, if joined by imaginary lines, may form a polygon (e.g., a quadrilateral shape) in which the internal angles at each vertex are different from one another. In an example, with respect to the positioning of the four markers, one of the internal angles may be greater than 180 degrees.

According to other example aspects, the UV disinfection system described herein may be applied to personal protection equipment (PPE). For example, the sensor tags may be attachable to PPE.

Aspects of the subject matter described herein may be implemented to realize one or more advantages. For example, the described techniques may support improved UV radiation coverage and improved power savings compared to some UV disinfection systems.

Aspects of the disclosure are initially described in the context of a UV disinfection system. Examples of processes, system operations, and sensor configurations that support a UV disinfection system with sensors and feedback are then described. Aspects of the disclosure are further illustrated by and described with reference to apparatus diagrams, system diagrams, and flowcharts that relate to a UV disinfection system with sensors and feedback.

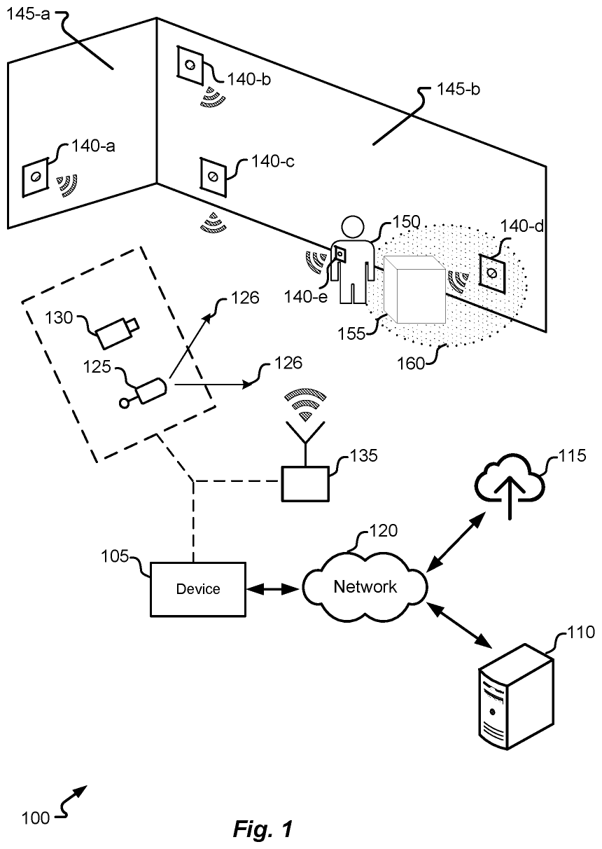

FIG. 1 illustrates an example of a system 100 that supports a UV disinfection system with sensors and feedback in accordance with aspects of the present disclosure. In some examples, the system 100 may be a UV disinfection system.

The system 100 may include a communication device 105 (or multiple communication devices 105), a server 110, a database 115, and a communication network 120. The communication device 105 may be referred to as a source wireless communication device. Non-limiting examples of the communication device 105 may include, for example, personal computing devices or mobile computing devices (e.g., laptop computers, mobile phones, smart phones, smart devices, wearable devices, tablets, etc.). In some examples, the communication device 105 may be operable by or carried by a human user. In some aspects, the communication device 105 may perform one or more operations autonomously or in combination with an input by the user, the communication device 105, and/or the server 110. In some aspects, the communication device 105 may be mounted on a transport instrument configured to patrol a target area.

The server 110 may be, for example, a cloud-based server. In some aspects, the server 110 may be a local server connected to the same network (e.g., LAN, WAN) associated with the communication device 105. The database 115 may be, for example, a cloud-based database. In some aspects, the database 115 may be a local database connected to the same network (e.g., LAN, WAN) associated with the communication device 105 and/or the server 110. The database 115 may be supportive of data analytics, machine learning, and AI processing.

The communication network 120 may facilitate machine-to-machine communications between any of the communication device 105 (or multiple communication device 105), the server 110, or one or more databases (e.g., database 115). The communication network 120 may include any type of known communication medium or collection of communication media and may use any type of protocols to transport messages between endpoints. The communication network 120 may include wired communications technologies, wireless communications technologies, or any combination thereof.

The Internet is an example of the communication network 120 that constitutes an Internet Protocol (IP) network consisting of multiple computers, computing networks, and other communication devices located in multiple locations, and components in the communication network 120 (e.g., computers, computing networks, communication devices) may be connected through one or more telephone systems and other means. Other examples of the communication network 120 may include, without limitation, a standard Plain Old Telephone System (POTS), an Integrated Services Digital Network (ISDN), the Public Switched Telephone Network (PSTN), a Local Area Network (LAN), a Wide Area Network (WAN), a wireless LAN (WLAN), a Session Initiation Protocol (SIP) network, a Voice over Internet Protocol (VoIP) network, a cellular network, and any other type of packet-switched or circuit-switched network known in the art. In some cases, the communication network 120 may include of any combination of networks or network types. In some aspects, the communication network 120 may include any combination of communication mediums such as coaxial cable, copper cable/wire, fiber-optic cable, or antennas for communicating data (e.g., transmitting/receiving data).

The system 100 may further include a radiation source 125, an image sensor 130, an RF transceiver 135 (also referred to herein as an RF transceiver device, an RF transponder device, or an RF transmitter-receiver device), and sensor tags 140 (e.g., sensor tag 140-a through sensor tag 140-d). In some aspects, the radiation source 125 and/or the image sensor 130 may be network capable devices capable of directly communicating with the communications network 120 (e.g., via a wired or wireless connection). For example, the radiation source 125 may be electrically coupled to the communication device 105 on a transport instrument configured to patrol a targeted area where the sensor tags 140 are located. In some other aspects, the radiation source 125 and/or the image sensor 130 may indirectly communicate with the communications network 120 (e.g., via the communication device 105).

The radiation source 125 may be a UV light source configured to emit light 126 (UV radiation) in the UV range. For example, the radiation source 125 may be configured to emit UV light associated with disinfecting the air and/or target surfaces 145 (e.g., target surface 145-a, target surface 145-b) of a physical environment to be sanitized by the system 100. The physical environment may include, for example, a hospital environment (e.g., a room in a hospital), a controlled environment (e.g., a clean room), a residential environment (e.g., a hotel room, a vacation rental), a commercial environment (e.g., a fitness facility, an office facility), or the like. In an example, the radiation source 125 may be configured to emit light having a wavelength between 100 nm to 280 nm (i.e., UV-C wavelength range).

The radiation source 125 may include a location sensor configured to record location information associated with the radiation source 125. In an example, the location sensor may be configured to record and output coordinates, positioning information, orientation information, velocity information, or the like. For example, the radiation source 125 may include an accelerometer, a GPS transponder, an RF transceiver, a gyroscopic sensor, or any combination thereof.

The image sensor 130 may be a single image sensor. In some aspects, the image sensor 130 may be an array of image sensors. The image sensor 130 may be integrated within the communication device 105 or a camera device. In an example, the image sensor 130 may be included in a standalone camera device or a camera device integrated with the communication device 105. The image sensor 130 may include photodiodes sensitive (e.g., capable of detecting) to light of a configured frequency band(s).

The camera device may be mechanically mounted to or within a housing of the communication device 105 in a manner that allows rotational degrees of freedom of the camera device and/or the image sensor 130. In another example, the camera device may be mounted to any surface or any object. In some aspects, the camera device may be a spherical camera device (e.g., for providing a spherical field of view).

The image sensor 130 may include a location sensor configured to record location information associated with the image sensor 130. In an example, the image sensor 130 may be configured to record and output coordinates, positioning information, orientation information, velocity information, or the like. For example, the image sensor 130 may include an accelerometer, a GPS transponder, an RF transceiver, a gyroscopic sensor, or any combination thereof.

The system 100 may include multiple cameras (and multiple image sensors 130). The multiple cameras (and multiple image sensors 130) may be integrated with the communication device 105 or multiple communication devices 105. In some aspects, the multiple cameras (and multiple image sensors 130) may be standalone cameras separate from the communication devices 105.

The image sensor 130 include any combination of photodiodes, photocathodes, and/or photomultipliers. The image sensor 130 may be configured to detect light within any defined wavelength range (e.g., visible spectrum, electromagnetic spectrum of ultraviolet radiation (UVR), etc.). In some aspects, the image sensor 130 may include one or more photodiodes implemented as UV sensors.

In an example, the image sensor 130 may be sensitive within the range of 200 nm to 1100 nanometers. In some examples, the image sensor 130 may include UV sensors that are sensitive to ultraviolet A (UV-A) radiation (e.g., having a wavelength from 315 nm to 400 nm), ultraviolet B (UV-B) radiation (e.g., having a wavelength from 280 nm to 315 nm), ultraviolet C (UV-C) radiation (e.g., having a wavelength from 100 nm to 280 nm), or any combination thereof.

The image sensor 130 and the radiation source 125 may be positioned or located within the system 100 such that, a relative position between the image sensor 130 and the radiation source 125 is configured or known (e.g., by the system 100, the communication device 105, the server 110, etc.). Based on the relative positioning, the image sensor 130 and the radiation source 125 may share a same frame of reference (e.g., a shared reference point).

In an example, any of the radiation source 125, the image sensor 130, and the RF transceiver 135 may be integrated within the communication device 105. For example, any of the radiation source 125, the image sensor 130, and the RF transceiver 135 may communicate within the communication device 105 over a system bus included in the communication device 105. In another example, any of the radiation source 125, the image sensor 130, and the RF transceiver 135 may be external to the communication device 105. For example, any of the radiation source 125, the image sensor 130, and the RF transceiver 135 may be electrically coupled (e.g., via a wired connection) to the communication device 105. In some examples, any of the radiation source 125, the image sensor 130, and the RF transceiver 135 may communicate with the communication device 105 via a wired connection, a wireless connection, or the communications network 120.

In some aspects, any of radiation source 125, the image sensor 130, and the RF transceiver 135 may be mechanically mounted to a transport instrument configured to move about the physical environment. In an example, movement of the transport instrument may be controlled by the system 100 (e.g., via commands by the communication device 105 or the server 110). In some other aspects, movement of the transport instrument may be autonomous or semi-autonomous (e.g., based on a schedule or programming). For example, the transport instrument may be instructed to patrol a target area associated with the physical environment. The transport instrument may be, for example, a mobile vehicle, a motorized robot, or the like.

The sensor tags 140 may be, for example, communication devices capable of transmitting and receiving signals (e.g., via wired or wireless communications). For example, the sensor tags 140 may communicate with the communication device 105 via an RF communications link established between the sensor tags 140 and the communication device 105. The sensor tags 140 may be referred to as sensor devices, receiver wireless communication devices, receiver devices, UV tags, or UV sensor tags.

Non-limiting examples of the sensor tags 140 may include, for example, Internet of Things (IoT) devices, wearable devices, or the like. For example, the sensor tags 140 may be disposed (e.g., attached, affixed, installed) on one or more target surfaces 145 (e.g., target surface 145-a, target surface 145-b) of the physical environment. In some other aspects, the sensor tags 140 may be operable by, carried by, or worn by a user 150. For example, a sensor tag 140 (e.g., sensor tag 140-e) may be attachable to PPE of a user 150. In some aspects, the sensor tags 140 may perform one or more operations autonomously or in combination with an input by the user, the communication device 105, the server 110, and/or a central controller.

Each of the sensor tags 140 may include one or more sensors. For example, each of the sensor tags 140 may include radiation sensors, light sensors, UV sensors, or electronic UV sensors. In an example, a sensor included in a sensor tag 140 may detect and measure UV radiation (e.g., UV-C radiation) emitted by the radiation source 125.

In some other examples, each of the sensor tags 140 may include a location sensor configured to record location information. In an example, the location sensor may be configured to record and output coordinates, positioning information, orientation information, velocity information, or the like. For example, the image sensor 130 may include an accelerometer, a GPS transponder, an RF transceiver, a gyroscopic sensor, or any combination thereof.

In some aspects, a sensor of a sensor tag 140 may convert detected UV radiation into digital data (e.g., a digital measurement value). In some examples, each sensor tag 140 may communicate measured UV radiation power at the sensor tag 140 (e.g., the total energy of UV radiation). Each sensor tag 140 may communicate the digital data (e.g., measured UV radiation power) to other devices in the system 100 using RF communications. For example, each sensor tag 140 may communicate the digital data to the communication device 105 (or other communication devices 105) via an RF transceiver included in the sensor tag 140. The sensor tag 140 may have an internal RF transceiver circuit configured to transmit data when the sensor tag 140 is within a visible range of the image sensor 130 (e.g., visible to the image sensor 130, within a threshold distance such that the sensor tag 140 is detectable by or visible to the image sensor 130). In this way, for example, data transmissions between the sensor tag 140 and the image sensor 130 can be performed with low energy (e.g., low transmission power) because the sensor tag 140 and the image sensor 130 (and the communication device 105) are in a close proximity (e.g., within a threshold distance of each other).

Based on digital data (e.g., digital measurement values) received or read from the sensor tags 140, the system 100 may calculate the total UV radiation (e.g., total energy) emitted by the radiation source 135 into the physical environment. In some aspects, the total UV radiation may include the total amount of UV radiation received at the sensor tags 140. In an example, based on the total amount of UV radiation received at the sensor tags 140, the system 100 may calculate or estimate the total UV radiation incident the physical environment (e.g., total UV radiation incident the target surfaces 145).

In some examples, a sensor tag 140 may be powered by an electrical power source electrically coupled to the sensor tag 140. For example, the electrical power source may include an external power source (e.g., an external power supply, a power outlet). In another example, the electrical power source may include batteries integrated with the sensor tag 140 (e.g., included in a housing of the sensor tag 140). The batteries may include long-life or extended-life batteries.

In some other aspects, the sensor tag 140 may be powered by energy harvesting techniques including solar, piezo-vibrational, or wireless charging. In an example, the sensor tag 140 may be powered by RF wireless waves (e.g., RF wireless charging). For example, the communication device 105 may wirelessly power or charge the sensor tag 140 using RF signals emitted from the RF transceiver 135. In some aspects, the system 100 may energize sensor tags 140 and sensors included therein (within an RF range of the RF transceiver 135), which may provide power to enable UV sensing operations of the sensor tags 140 and sensors. In some other aspects, the sensor tags 140 may be energized by background RF signals (e.g., Bluetooth, WiFi, 3G, 4G, or 5G sources) associated with the physical environment and/or RF signals within a threshold distance of the sensor tags 140.

Each sensor tag 140 may include multiple markers. The system 100 (e.g., communication device 105) may detect the markers of the sensor tag 140 using the image sensor 130, and based on the detected markers, the system 100 may identify location information of the sensor tag 140 (or a radiation sensor included in the sensor tag 140). For example, the system 100 may use the location information to estimate the distance between the image sensor 130 and the sensor tag 140. In another example, the system 100 may calculate a viewing angle and/or distance from the image sensor 130 to the sensor tag 140. Based on the relative known positioning between the image sensor 130 and the radiation source 125, the system 100 may calculate an angle and/or distance from the radiation source 125 to the sensor tag 140.

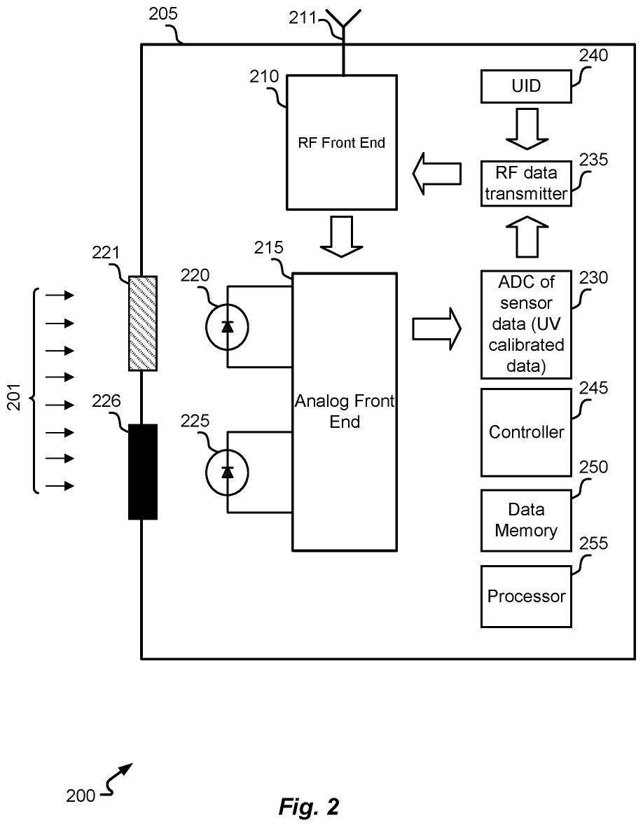

In some aspects, the markers may be UV luminous markers. For example, the markers may be formed of a radiation sensitive material disposed on a housing of the sensor tag 140. In an example, the radiation sensitive material may react (e.g., change color) in response to UV light emitted by the radiation source 125 (e.g., light having a wavelength in the UV-C wavelength range). For example, the radiation sensitive material may transition between different colors based on an exposure to the UV light emitted by the radiation source. Example aspects of the sensor tags 140, the markers, and components and functionalities of the sensor tags 140 are further described with reference to FIGS. 2 through 6.

In some aspects, the image sensor 130 may capture images of the physical environment inclusive of the sensor tags 140 and/or target surfaces 145. The system 100 may use one or more captured images (e.g., captured images of the physical environment) as a reference for determining characteristics associated with the physical environment.

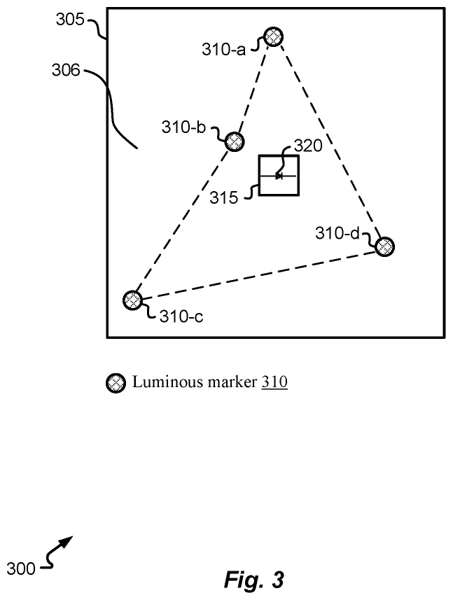

For example, the image sensor 130 may visually detect the sensor tags 140 based on the captured images. In some cases, the image sensor 130 may visually detect the sensor tags 140 based on radiation sensitive material (e.g., visual markers) disposed on the sensor tags 140. Example aspects of the radiation sensitive material and visual markers are described with reference to FIG. 3 through FIG. 6.

Based on the captured images, the system 100 may determine characteristics of sensor tags 140 within the physical environment (e.g., distance from the image sensor 130 and/or the radiation source 125 to the sensor tags). For example, the system 100 may determine location information, orientation information, velocity information (e.g., for examples in which an sensor tag 140 is attached to a moving object or a user), and/or identification information associated with the sensor tags 140. In some examples, the system 100 may determine characteristics of physical objects within the physical environment (e.g., distance from the image sensor 130 and/or the radiation source 125 to the physical objects).

In some other examples, the system 100 (e.g., using the image sensor 130) may detect and record geolocation information (e.g., coordinates, GPS) coordinates) of the sensor tags 140 with respect to the physical environment. For example, the system 100 may detect and record the geolocation information based on location information of the image sensor 130 and the calculated angle and distance from the image sensor 130 to the sensor tag 140. In some aspects, the system 100 may detect and record the geolocation information based on location information of the communication device 105 (when the image sensor 130 is integrated with the communication device 105).

The communication device 105 may communicate (e.g., transmit or receive) data packets with one or more other devices of the system 100. For example, the communication device 105 may exchange data packets with another communication device 105, the server 110, the database 115, the radiation source 125, the image sensor 130, or the RF transceiver 135, or the via the communication network 120. In some examples, the communication device 105 may communicate with another device (e.g., another communication device 105, database 115, radiation source 125, image sensor 130, RF transceiver 135) via the server 110.

The system 100 may support a global wake-up of the sensor tags 140 (e.g., wake-up of sensors included in the sensor tags 140). The sensor tags 140 (and sensors) may be, for example, located within a RF coverage area of the system 100. In some aspects, the RF coverage area may be based on RF transmission power of the RF transceiver 135, RF signal quality of a communications link (e.g., an RF communications link) established between the RF transceiver 135 and the sensor tags 140 and/or a distance threshold from the RF transceiver 135.

In some aspects, the system 100 may support temporal synchronization among the sensor tags 140. For example, the communication device 105 (e.g., via the RF transceiver 135) may transmit temporal reference data (e.g., time data) to all the sensor tags 140 (e.g., periodically, based on a schedule, on demand, as part of a global wake-up event, etc.). The sensor tags 140 may synchronize with the temporal reference data. In an example, each sensor tag 140 (e.g., sensor tag 140-a through sensor tag 140-d) may report UV levels measured by an included sensor, in combination with temporal information (e.g., timestamp data) corresponding to the measurements. The timestamp data may be synchronized with the temporal reference data.

In some aspects, using the RF transceiver 135, the communication device 105 may read digital data generated by each sensor tag 140. The sensor tags 140 may communicate respective digital data to the communication device 105, for example, over an established communications link or via broadcast communications. In an example, the digital data may include UV radiation information and time information. For example, the digital data may include measured UV radiation levels and timestamps corresponding to when the UV radiation levels are measured by a sensor tag 140 and/or read by the communication device 105. In some examples, the sensor tags 140 may be assigned respective unique identifiers, and the UV disinfection system may reference the identifiers to distinguish between UV data respectively measured by each sensor tag 410.

According to example aspects of the present disclosure, the system 100 may emit UV radiation directed toward a target surface 145. In an example, the system 100 may identify sensor tags 140 and respective sensor tag locations through visual means (e.g., based on images captured by the image sensor 130 and/or detection of respective markers). For example, for each sensor tag 140, the system 100 may support visual calculation and estimation of location information (e.g., coordinates, positioning) of the respective markers. The system 100 may determine direction and distance of the markers (and corresponding sensor tags 140), for example, in relation to the radiation source 125 and/or the image sensor 130.

Based on the location information of the markers (and corresponding sensor tags 140), the system 100 may apply RF beamforming techniques for directing RF signals from the communication device 105 to the sensor tags 140. In some cases, based on the location information, the system 100 may apply the RF beamforming techniques to establish a communications link (e.g., a directed RF link) between the communication device 105 and the sensor tags 140. In some aspects, the directed RF link may support improved RF energy transfer (e.g., higher efficiency) to the sensor tags 140. In some other aspects, the directed RF link may support improved RF data communications (e.g., increased RF communications quality, increased QoS) between the communication device 105 and the sensor tags 140.

The system 100 may collect and aggregate data from the sensor tags 140, in combination with identification information associated with the sensor tags 140. The identification information may include, for example, unique identifiers (UIDs) associated with the sensor tags 140. In some examples, the data may include measured UV radiation levels and corresponding temporal information. Based on aggregating the data, the system 100 may map topological information (e.g., location information, orientation information) associated with the sensor tags 140.

In some aspects, based on the aggregated data, the system 100 may determine the amount of UV light incident a physical environment and/or any target surface 145 of the physical environment. For example, the system 100 may determine the amount of surface radiation (e.g., UV radiation coverage) and time duration (e.g., radiation duration) with respect to the physical environment and/or target surfaces 145. In some aspects, the system 100 may process the aggregated data using any combination of data analytics, machine learning and AI processing. In some examples, the system 100 may transfer the aggregated data or any portion thereof to cloud-based data storage or the server 110 via wired or wireless communication.

A machine learning network (e.g., implemented in the system 100, the communication device 105, or the server 110) may evaluate the data and generate feedback information (e.g., probability information, confidence information) with respect to the UV radiation coverage with respect to the physical environment and/or target surface. For example, the machine learning network may generate and output feedback information with respect to the amount of UV radiation energy detected by radiation sensors of a sensor tag 140. In some aspects, the feedback information may be associated with the total amount of UV radiation energy detected by radiation sensors of multiple sensor tags 140 (e.g., all sensor tags 140 included in the physical environment). In some cases, based on an evaluation of the data, the machine learning network may predict the amount of UV radiation energy incident one or more target surfaces in the physical environment.

In an example, based on the aggregated data and/or images captured by the image sensor 130, the machine learning network may output predicted radiation coverage corresponding to a target area of the physical environment and a temporal period. In some examples, the output may include probability information corresponding to the predicted radiation coverage and/or confidence information associated with the probability information. In some aspects, the system 100 (e.g., using the machine learning network) may calculate or suggest relatively shorter temporal periods for applying UV radiation compared to some other UV disinfection systems.

In another example, based on the aggregated data and/or images captured by the image sensor 130, the machine learning network may output predicted location information and/or predicted movement associated with the sensor tags 140 and a predicted temporal period. In some aspects, the output may include predicted orientation information associated with the sensor tags 140 and the predicted temporal period. In some other aspects, the output may include predicted velocity information associated with the sensor tags 140 and the predicted temporal period. The output may include probability information and/or confidence information associated with the predicted location information, the predicted orientation information, the predicted velocity information, and/or the predicted temporal period.

Based on the output from the machine learning network, the system 100 (e.g., central controller) may control a location of the radiation source 125, an emission direction of the radiation source 125, an emission power of the radiation source 125, and/or an emission duration of the radiation source 125.

The system 100 may collect and provide UV data to the machine learning network periodically (e.g., based on a schedule, a temporal duration, etc.) or in real-time. In some examples, based on the feedback information provided by the machine learning network, the system 100 may set or adjust one or more parameters of the radiation source 125. For example, the system 100 may adjust the output power of the radiation source 125, reposition the radiation source 125 (e.g., modify an emission direction of the radiation source 125), or modify a location of the radiation source 125 based on the feedback information.

In some aspects, the system 100 may aggregate location information of the sensor tags 140. The system 100 (or the machine learning network) may analyze the location information, for example, to verify the operation of the sensor tags 140. For example, the system 100 may identify whether any sensor tags 140 have been tampered with, incorrectly located by the system 100 (e.g., not detected), or damaged. In some aspects, the system 100 may identify whether any sensor tags 140 are non-visible to the radiation source 125. For example, the system 100 may identify whether an object located between the radiation source 125 and a sensor tag is preventing UV light emitted by the radiation source 125 from reaching the sensor tag 140.

The system 100 may support synchronization between the communication device 105 and the sensor tags 140 (e.g., time synchronization) in combination with various system level functionality. In some aspects, the system 100 may support synchronization between any of the communication device 105, the RF transceiver 135 of the communication device 105, the radiation source 125, the image sensor 130, and the sensor tags 140 in combination with system level functionality. For example, the system 100 may support control (e.g., via commands, signals, etc.) of any of the communication device 105, the RF transceiver 135 of the communication device 105, the radiation source 125, the image sensor 130, and the sensor tags 140.

In an example, the system 100 may support a central controller (e.g., a central processing device). In some aspects, the central controller may be implemented at the communication device 105 or the server 110 of the system 100. For example, the central controller may be included in a processor of the communication device 105 or the server 110. In an example, the central controller may control multiple devices (e.g., communication devices 105, servers 110, radiation source 125, image sensor 130, sensor tags 140, etc.) included in the system 100, where each of the devices is associated with a respective function or task. In some cases, each of the devices may transmit status information and/or data to the central controller. In some aspects, each of the devices may receive actuating signals from the central controller in association with performing a respective function or task.

In an example in which the central controller is implemented at the server 110, the central controller may communicate with another communication device 105, another server 110, the radiation source 125, the image sensor 130, or the sensor tags 140 via the communication device 105 and/or the communications network 120. Example aspects of the system 100 described herein may be performed by the central controller in combination with devices of the system 100.

In an example, the system 100 (e.g., via the central controller) may support aggregation of the intensity information recorded by the sensor tags 140. In some examples, the system 100 may maintain a record of the intensity information recorded by the sensor tags 140. The system 100 may use the intensity information to compute information indicative of whether the target area is sufficiently disinfected (e.g., the UV radiation at the target area satisfies a disinfection level or a disinfection coverage, a pathogen level at the target area satisfies a threshold, etc.). In some aspects, the sensor tags 140 may transmit the intensity information to the central controller wirelessly (e.g., via an RF communications link). The intensity information may form a portion of a safety control sub-system of the system 100. For example, when a human subject is present within the target area, the system 100 may be configured to output a notification (e.g., sound an alarm) to warn the human subject about the safety level of the radiation (e.g., based on a detected intensity information exceeding a threshold).

In some aspects, the system 100 may evaluate one or more parameters associated with the radiation source 125 based on the aggregated intensity information. In some aspects, the one or more parameters may include a degradation level associated with the UV radiation emitted by the radiation source 125, a degradation rate associated with the emitted UV radiation, or both. In some aspects, the radiation source 125 may degrade over time (e.g., based on usage, manufacturing age, etc.), and when the degradation exceeds a predetermined level, the radiation source 125 may have to be replaced for being unable to sufficiently disinfect the entire target area. In some other aspects, the one or more parameters associated may include a quality level of a disinfection session implemented by the system 100. In some cases, the sensor tags 140 output a notification associated with the quality level of the disinfection session compared to a quality level threshold.

In some aspects, the system 100 (e.g., via the central controller) may support controlling the emission of UV radiation based on the data aggregated from the sensor tags 140 (e.g., as measured by the sensors). For example, the system 100 may position the radiation source 125 and/or direct emissions of the radiation source 125 based on the data. In another example, the system 100 may position and/or direct the RF transceiver 135 of the communication device 105 based on the data aggregated from the sensor tags 140 (e.g., aggregated intensity information) and image data captured by the image sensor 130 (e.g., visual confirmation information with respect to sensor tag locations). Accordingly, for example, the system 100 may control a location of the radiation source 125, an emission direction of the radiation source 125, an emission power of the radiation source 125, or a combination thereof.

In some aspects, the sensor tags 140 may record temporal information (e.g., timestamp values) associated with the intensity information measured by sensors included in the sensor tags 140. In an example, the system 100 may evaluate the one or more parameters associated with the radiation source 125 based on the temporal information.

In some examples, the system 100 may support aggregation of intensity information from multiple sensor tags 140 based on temporal information (e.g., timestamp values) associated with the intensity information. For example, the sensor tag 140-a may record first intensity information associated with UV radiation received at the sensor tag 140-a, along with a timestamp value associated with the first intensity information. The sensor tag 140-b may record second intensity information associated with UV radiation received at the sensor tag 140-b, along with a timestamp value associated with the second intensity information. The system 100 may aggregate the first intensity information and the second intensity information based on comparison of the timestamp values. For example, the system 100 may aggregate the first intensity information and the second intensity information based on whether a difference between the respective timestamp values is below a temporal threshold.

According to other example aspects of the present disclosure, the central controller may be configured to receive interrupt signals from the sensor tags 140. For example, each sensor tag 140 may generate an interrupt signal when UV radiation measured at the sensor tag 140 (e.g., radiation intensity level measured at a radiation sensor of the sensor tag 140) is above a predetermined overlimit level.

In an example, the sensor tag 140-e (attached to PPE of the user 150) may generate and transmit an interrupt signal (e.g., a pause command, an indicator) to the central controller when UV radiation measured at the sensor tag 140-e is above a predetermined safety level associated with human exposure to the UV radiation. In response to the interrupt signal, the central controller may pause UV radiation transmissions (e.g., for a temporal period, intermittently, etc.) by the radiation source 125 and/or reduce emission power until the UV radiation measured at the sensor tag 140-e is below the predetermined safety level. In an example, the sensor tag 140-e may transmit a signal (e.g., a resume command, an indicator) to the central controller to continue UV radiation transmissions by the radiation source 125 and/or increase emission power (e.g., to a configured power level, a previous power level, etc.).

In some aspects, the sensor tag 140-e may directly alert the user 150 that the measured UV radiation is above the predetermined safety level. For example, the sensor tag 140-e may generate and output any combination of audible (e.g., via a speaker), visual (e.g., via a display, a light emitting diode (LED), or a color transition of a marker disposed on the sensor tag 140-e), or haptic notifications for alerting the user. In another example, the sensor tag 140-e may indirectly alert the user 150 via the central controller. For example, the sensor tag 140-e may transmit the interrupt signal to the central controller, and the central controller may control the communication device 105 to alert the user 150.

For example, the communication device 105 may be a mobile device worn or carried by the user 150, and the communication device 105 may output an audible, visual, and/or haptic notification to the user 150. In some other aspects, the sensor tag 140-e may passively alert the user 150 via a radiation sensitive material disposed on the sensor tag 140-e. Example aspects and characteristics of the radiation sensitive material and an sensor tag 140 are described with reference to FIG. 3.

In another example, a sensor tag 140 may generate and transmit a signal (e.g., a pause command, a resume command, an indicator) based on a pathogen level detected by the sensor tag 140 with respect to a threshold. In response to the signal, the central controller may interrupt UV radiation transmissions (e.g., based on a pause command), resume UV radiation transmissions (e.g., based on a resume command), increase emission power of the radiation source 125 (e.g., based on an indication that pathogen levels are above a threshold), and/or increase the duration for emitting UV radiation from the radiation source 125 (e.g., based on an indication that pathogen levels are above a threshold).

In some aspects, the central controller may evaluate the radiation source 125 using measured UV radiation levels (also referred to herein as measured UV intensity or intensity information) provided by sensor tags 140 that are visible to the image sensor 130. In some examples, the central controller may position or relocate the image sensor 130 (e.g., using a transport instrument coupled to the image sensor 130) for cases in which a physical object between the image sensor 130 and a sensor tag 140 prevents the image sensor 130 from visually detecting the sensor tag 140.

For example, an object 155 may be located between the image sensor 130 and the sensor tag 140-d such that the sensor tag 140-d is not visible to the image sensor 130. Based on previously recorded UV radiation measurements and/or images previously captured by the image sensor 130, the central controller may be aware of the presence of the sensor tag 140-d. However, based on a current image (e.g., a static image, a video image) captured by the image sensor 130, the central controller may identify that the sensor tag 140-d is absent from a visual zone 160 associated with the captured image. The central controller may position or relocate the image sensor 130 (e.g., using the transport instrument coupled to the image sensor 130) until the image sensor 130 visually detects the sensor tag 140-d. Once the sensor tag 140-d is visually detected, the central controller may evaluate the radiation source 125 using the UV radiation measurements from the sensor tag 140-d.

In another example, the object 155 may be located between the RF transceiver 135 and the sensor tag 140-d such that RF communications between the RF transceiver 135 and the sensor tag 140-d are obstructed. Based on previously recorded UV radiation measurements received by the RF transceiver 135 and/or images previously captured by the image sensor 130, the central controller may be aware of the presence of the sensor tag 140-d. However, based on a current reading via the RF transceiver 135, the central controller may identify that RF communications between the RF transceiver 135 and the sensor tag 140-d are obstructed. The central controller may position or relocate the RF transceiver 135 (e.g., using the transport instrument coupled to the RF transceiver 135) until an RF communications link is reestablished with the sensor tag 140-d.

In some other aspects, the central controller may compute a distance between the radiation source 125 (or image sensor 130) and a sensor tag 140 using the example techniques described herein. The central controller may compare the distance to the UV radiation levels measured at the sensor tag 140 so as to compute an evaluation result regarding the radiation source 125 (e.g., UV radiation intensity, UV radiation power, UV radiation coverage, UV radiation transmission efficiency, etc.). In some examples, the evaluation result may include degradation information of the radiation source 125. In some other examples, the evaluation result may include an evaluation result of whether an area adjacent to a sensor tag 140 is sufficiently disinfected (e.g., the amount of pathogens present on a target surface 145 on which a sensor tag 140 is disposed is below a threshold).

In some cases, the central controller may position and/or relocate the radiation source 125 (e.g., using a transport instrument coupled to the radiation source 125) based on a predetermined threshold corresponding to a UV radiation intensity for removing pathogens from the physical environment (e.g., a target area, a target surface 145-a). The predetermined threshold may be referred to as a germ-kill limit. In an example, the central controller may position and/or relocate the radiation source 125 such that a distance between the sensor tag 140-a and the radiation source 125 is reduced. The central controller may adjust the location, an emission direction, and/or an emission power of the radiation source 125, for example, until the measured UV radiation at the sensor tag 140-a (and thereby the UV radiation at the target surface 145-a) exceeds the predetermined threshold. In some cases, the central controller may pause (or unpause) UV emissions and/or reduce (or increase) emission power of the radiation source 125 based on a comparison of the measured UV radiation at the sensor tag 140-a (and thereby the UV radiation at the target surface 145-a) to the predetermined threshold.

In some aspects, the central controller may maintain location records of the sensor tags 140 in the physical environment. The central controller may generate and/or update the location records based on images captured by the image sensor 130 and/or UV radiation measurements reported to the central controller by the sensor tags 140. In some aspects, based on the captured images and/or UV radiation measurements (or the location records generated therefrom), the central controller may identify whether any of the sensor tags 140 are inactive, damaged, not present in the physical environment, etc. The central controller may alert the user 150 (or any components of the system 100) of any sensor tags 140 that the central controller identifies as inactive, damaged, or not present in the physical environment.

While the illustrative aspects, embodiments, and/or configurations illustrated herein show the various components of the system 100 collocated, certain components of the system 100 can be located remotely, at distant portions of a distributed network, such as a Local Area Network (LAN) and/or the Internet, or within a dedicated system. Thus, it should be appreciated, that the components of the system 100 can be combined in to one or more devices or collocated on a particular node of a distributed network, such as an analog and/or digital telecommunications network, a packet-switched network, or a circuit-switched network. It will be appreciated from the following description, and for reasons of computational efficiency, that the components of the system 100 can be arranged at any location within a distributed network of components without affecting the operation of the system 100.

FIG. 2 illustrates an example block diagram 200 of a sensor tag 205 that supports a UV disinfection system with sensors and feedback in accordance with aspects of the present disclosure. The sensor tag 205 may receive incident light 201 of any wavelength (e.g., ambient light, UV light, etc.) as described herein. In some examples, the sensor tag 205 may include an RF front end 210, an antenna 211, an analog front end (AFE) 215 (also referred to herein as a sensor AFE 215), a sensor 220, a UV bandpass filter 221, a sensor 225, an opaque dark reference 226, an ADC 230, an RF data transmitter 235, a unique identifier 240, a controller 245, data memory 250, and a processor 255. The sensor tag 205 may include aspects of like elements described with reference to FIG. 1.

The RF front end 210 may support energy harvesting and RF data communications via the antenna 211. For example, the RF front end 210 may support energy harvesting associated with RF signals emitted by the RF transceiver 135 (described with reference to FIG. 1) and/or background RF signals (e.g., Bluetooth, WiFi, 3G, 4G, or 5G sources) associated with a physical environment.

In an example, the sensor tag 205 (or one or more components included in the sensor tag 205) may enter an awake state (an active state) when an amount of RF energy received at the RF front end 210 (e.g., extracted RF energy) is greater than or equal to a threshold. In some aspects, the AFE 215 may generate a wake-up signal when the RF energy is greater than or equal to the threshold. In some aspects, in the awake state, the sensor tag 205 may operate in an ultra-low power mode. For example, the sensor tag 205 may implement ultra-low power sensor operations including dark current mitigation (e.g., cancellation of dark current described herein), data conversion (e.g., ADC conversion) of measured UV intensity, data calibration, and data storage.

In another aspect, the sensor tag 205 may enter the awake state based on solar charging of the sensor tag 205. In an example, the sensor tag 205 may include a sensor (not illustrated) that is sensitive to (e.g., detects) light having a wavelength between 380 nm to 780 nm (e.g., ambient light). In some aspects, the AFE 215 may generate a wake-up signal if a wake-up condition is detected by the ambient sensor. For example, the AFE 215 may generate the wake-up signal if an amount of ambient light detected by the sensor satisfies a threshold. In some aspects, the AFE 215 may include a wake-up device (not illustrated) configured to generate the wake-up signal. The wake-up device may include, for example, a combination of hardware components (e.g., logic circuitry, wiring, etc.).

The antenna 211 may be, for example, a tag-printed coil antenna. In some aspects, the sensor tag 205 may include one or more antennas 211. The antenna 211 may be electrically coupled to the RF front end 210.

The AFE 215 may be a signal conditioning chip that enables a direct connection between the sensor 220 and sensor 225 to the signal processing components of the sensor tag 205 (e.g., ADC 230, processor 255). In some aspects, the AFE 215 may receive current or voltage levels output by the sensor 220 and sensor 225.

The sensor 220 may be an image sensor, for example, a UV sensor that is sensitive to UV-A radiation (e.g., having a wavelength from 315 nm to 400 nm), UV-B radiation (e.g., having a wavelength from 280 nm to 315 nm), UV-C radiation (e.g., having a wavelength from 100 nm to 280 nm), or any combination thereof. In an example, the sensor 220 may be a photodiode that is sensitive to (e.g., capable of detecting, responds to) UV-C radiation.

The UV bandpass filter 221 may be positioned at an opening (e.g., a sensor window) of a housing of the sensor tag 205. In an example, the UV bandpass filter 221 may be configured to pass UV radiation of a UV frequency band. For example, the UV bandpass filter 221 may be configured to pass UV radiation of the UV-C frequency band (e.g., 100 nm to 280 nm). UV radiation which passes through the UV bandpass filter 221 may be incident the sensor 220.

The sensor 225 may be a dark current compensation diode (also referred to herein as a dark current sensor) for dark current suppression. The opaque dark reference 226 may be positioned at another opening (e.g., sensor window) of the housing of the sensor tag 205. Light which passes through the opaque dark reference 226 may be incident the sensor 225. In an example, the AFE 215 may apply photocurrent generated by the sensor 225 to compensate for dark current generated at the sensor 220. Dark current at the sensor 220, for example, may include current generated at the sensor 220 in the absence of visible light.

In some aspects, the AFE 215 may calibrate data measured by the sensor 220 using current output at the sensor 225. The data measured by the sensor 220 may include, for example, UV radiation intensity (also referred to herein as intensity information or intensity level information). In some aspects, the AFE 215 may calibrate the data using the photocurrent generated by the sensor 225 (e.g., dark current compensation). In some aspects, the AFE 215 may include a calibration device (not shown) configured to calibrate the data. In some aspects, the calibration device may include or be coupled to the sensor 225. The calibration device may include, for example, a combination of hardware components (e.g., logic circuitry, wiring, etc.).

The ADC 220 may support data conversion (e.g., analog-to-digital conversion) of the calibrated data output by the AFE 215. The ADC 220 may output the converted data to the RF data transmitter 235.

The RF data transmitter 235 may support the transmission and reception of signals to and from the sensor tag 205. For example, the RF data transmitter 235 may generate modulated RF signals for carrying the data output by the ADC 230. In some aspects, the RF data transmitter 235 may append identification information associated with the unique identifier 240 to the data to distinguish the data from that of different sensor tags (e.g., another sensor tag 205). In some cases, the RF data transmitter 235 may demodulate RF signals received by the sensor tag 205.

The controller 245 may be located on the same chip (or a different chip) as the sensor tag 205. The controller 245 may instruct components in the sensor tag 205 (e.g., AFE 215, ADC 230, RF data transmitter 235, processor 255) to perform one or more processes and/or signal processing operations (e.g., dark current, data conversion, data calibration, data storage, signal conversion, signal amplification, etc.). In some examples, the controller 245 may be a programmed microprocessor or microcontroller. In some aspects, the controller 245 may include one or more CPUs, memory, and programmable I/O peripherals.

The data memory 250 may store sensor data (e.g., calibrated measurement data) provided by the AFE 215. In some aspects, the data memory 250 may store temporal information (e.g., timestamp data) corresponding to the measurement data. In some other aspects, the data memory 250 may store temporal reference data (e.g., time data) common to the sensor tag 205 and the system 100 of FIG. 1 (e.g., temporal reference data provided by a communication device 105 of FIG. 1).

The processor 255 may correspond to one or many computer processing devices. For example, the processor 255 may include a silicon chip, such as a Field Programmable Gate Array (FPGA), an Application-Specific Integrated Circuit (ASIC), any other type of Integrated Circuit (IC) chip, a collection of IC chips, or the like. In some aspects, the processors may include a microprocessor, a Central Processing Unit (CPU), a Graphics Processing Unit (GPU), or plurality of microprocessors configured to execute the instructions sets stored in a corresponding memory (e.g., memory 250). For example, upon executing the instruction sets stored in memory 250, the processor 255 may enable or perform one or more functions of the sensor tag 205.

In some examples, components described herein of the sensor tag 205 may communicate over a system bus (e.g., control busses, address busses, data busses, etc.) or wiring (e.g., traces) included in the sensor tag 205.

FIG. 3 illustrates an example 300 of a sensor tag 305 that supports a UV disinfection system with sensors and feedback in accordance with aspects of the present disclosure. The example 300 illustrates, for example, an external surface 306 of a housing of the sensor tag 305. The sensor tag 305 may include markers 310 (e.g., marker 310-a through marker 310-d), a sensor window 315, and a sensor 320. The sensor tag 305, the markers 310, the sensor window 315, and the sensor 320 may include aspects of like elements described with reference to FIGS. 1 and 2.

In some aspects, the markers 310 may be UV luminous markers. For example, the markers 310 may be formed of a radiation sensitive material disposed on the external surface 306. The radiation sensitive material may be, for example, a UV coating (e.g., a UV luminous paint). In an example, the radiation sensitive material may react (e.g., change color) in response to light having a wavelength in a UV wavelength range (e.g., 100 nm to 400 nm). In some examples, the radiation sensitive material may react in response to light having a wavelength in the UV-C wavelength range (e.g., UV-C light emitted by the radiation source 125 described with reference to FIG. 1). For example, the radiation sensitive material may transition between different colors based on an exposure to the UV-C light.

The radiation sensitive material, for example, may be colorless (e.g., transparent) outside of UV light conditions. For example, the radiation sensitive material, for example, may be colorless under ambient light conditions such as sunlight. In some aspects, the radiation sensitive material may react (e.g., change color) in response to UV light of different wavelengths. For example, the radiation sensitive material may change color to red, yellow, black, or green based in response to a wavelength associated with the UV light. In some cases, the radiation sensitive material may include any combination of fluorescent inks (e.g., sensitive to light having a wavelength of 200 nm to 400 nm), invisible inks (e.g., sensitive to light having a wavelength of 400 nm to 800 nm), ‘short wavelength ink’ sensitive to light having a wavelength of 254 nm), and ‘long wavelength ink’ sensitive to light having a wavelength of 254 nm and light having a wavelength of 365 nm).

In some examples, the sensor tag 305 may include a minimum of four markers 310 (e.g., marker 310-a through marker 310-d). In some cases, a set of markers 310 may be referred to as an object. For example, marker 310-a through marker 310-d may be referred to as a four-point object or a four-point visible marker. In an example, the markers 310 may represent four respective points (e.g., X-axis and Y-axis coordinates) in the Cartesian plane.

In some aspects, the markers 310 (e.g., a four-point object or a four-point visible marker) may be asymmetric with respect to a two-dimensional plane. For example, the markers 310 may be asymmetric with respect to any axis (or all axes) associated with the sensor tag 305. The asymmetry, for example, may prevent an image sensor (e.g., the image sensor 130 described with reference to FIG. 1) from capturing the same reference image when capturing images of the sensor tag 305 from different locations or perspectives. For example, the asymmetry may prevent the image sensor from capturing multiple images of the sensor tag 305 in which the markers 310 have the same marker positions, same marker sizes, or same distances therebetween.

In an example of aspects of the present disclosure, the markers 310 may be positioned on the sensor tag 305 such that the markers 310 are not located along shapes having an axis of symmetry. For example, the markers 310 may be positioned asymmetric to one another, forming a four-point object without an axis of symmetry. In an example, markers 310, if joined by imaginary lines (represented by dotted lines in FIG. 3), may form a polygon in which the internal angles at each vertex are different from one another. The polygon may be, for example, a quadrilateral shape. In an example, with respect to the positioning of the markers 310, one of the internal angles (e.g., an internal angle at marker 310-b) may be greater than 180 degrees.

In reference to FIG. 1 and FIG. 3, the system 100 (e.g., a communication device 105, the system 100, a central controller) may identify and locate the sensor tag 305 using the image sensor 130, based on the markers 310. For example, the image sensor 130 may distinguish the sensor tag 305 from other reference objects in a physical environment, based on the markers 310. For example, the image sensor 130 may identify the markers 310 based on a response of the markers 310 (e.g., a response of the respective UV coatings) to UV light emitted by the radiation source 125.

The image sensor 130 may capture an image of the sensor tag 305, where four points in the captured image correspond to the marker 310-a through marker 310-d. Based on the four points in the captured image, the system 100 (or machine learning network) may calculate or estimate the angle and distance between the image sensor 130 (or the radiation source 125) and the sensor tag 305. In some aspects, based on the four points and the captured image, the system 100 (or machine learning network) may calculate or estimate the angle and distance between the image sensor 130 (or the radiation source 125) and one or more objects in the captured image (e.g., objects in a physical environment). The system may support adjusting the resolution of the image sensor 130 and/or optical zoom settings to increase the accuracy with respect to calculated distance and angle.

The system 100 may detect and record geolocation information of the sensor tag 305 based on location information of the image sensor 130 and the calculated angle and distance between the image sensor 130 to the sensor tag 140. In some aspects, the system 100 may detect and record the geolocation information based on the location information of the communication device 105 (when the image sensor 130 is integrated with the communication device 105).

In some other aspects, the system 100 may determine an orientation of the sensor tag 305 and/or the sensor 320 through reference of the four-point object (e.g., quadrilateral shape) to a reference object. In some examples, the system 100 may determine the orientation through reference to a reference image including the reference object. In some examples, the system 100 may compare each side of the four-point object (e.g., quadrilateral shape) to the reference object (or reference image including the reference object) to compute rotational information of the sensor tag 305 and/or the sensor 320.

In some examples, the system 100 may compare one or more sides of the four-point object (e.g., quadrilateral shape) to one or more sides of the reference object (or reference image including the reference object) to compute distance information of the sensor tag 305 and/or the sensor 320 relative to the radiation source 125. In an example, the system 100 may compare all sides of the four-point object (e.g., quadrilateral shape) to all sides of the reference object (or reference image including the reference object) to compute the distance information.

In some cases, the system 100 may compare one or more sides of the four-point object to one or more sides of the reference object (or reference image including the reference object) to compute angular information of a surface of the sensor tag 305 relative to a beam axis (e.g., a boresight axis) of the radiation source 125.

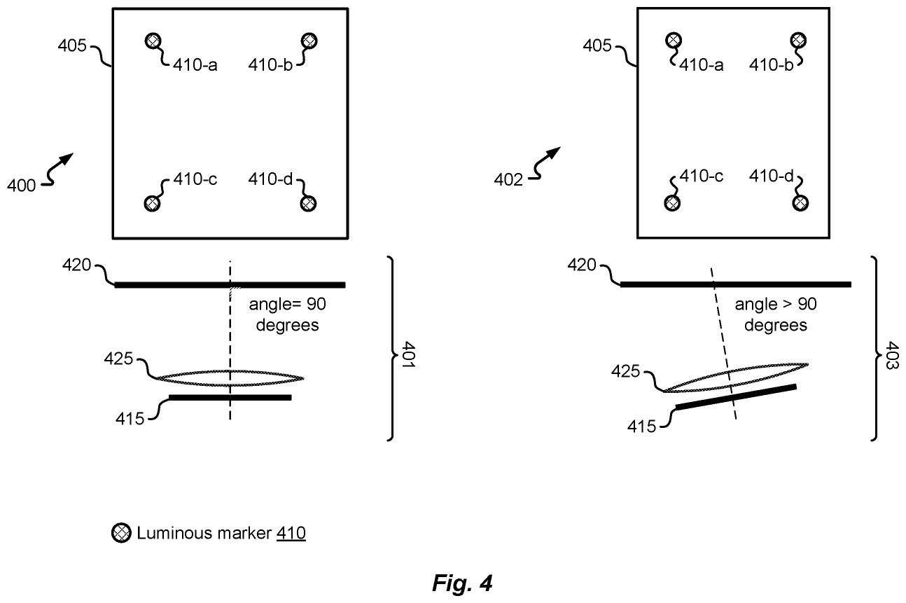

FIG. 4 illustrates example images 400 and 402 of a sensor tag 405 that supports a UV disinfection system with sensors and feedback in accordance with aspects of the present disclosure.

The example images 400 and 402 illustrate, for example, images captured by an image sensor 425. Referring to FIG. 4, the image sensor 425 is between an image sensor plane 415 (of the image sensor 425) and a tag plane 420 of the sensor tag 405. The sensor tag 405 may include markers 410 (e.g., marker 410-a through marker 410-d). The sensor tag 405, markers 410, and image sensor 425 may include aspects of like elements described with reference to FIGS. 1 through 3.

Image 400 illustrates an example of a captured image according to an example angular relationship 401, in which the image sensor plane 415 of the image sensor 425 is parallel to the tag plane 420 of the sensor tag 405. Image 402 illustrates an example of a captured image according to an example angular relationship 403, in which the image sensor plane 415 of the image sensor 425 is not parallel to the tag plane 420 of the sensor tag 405.

In some aspects, based on relative distances between the marker 410-a through marker 410-d in the images 400 and 402, the system 100 (e.g., communication device 105, image sensor 130, central controller) may estimate an angle of view between the image sensor 425 and the sensor tag 405. For example, the system 100 may estimate an angle of view in a two-dimensional plane. In some aspects, utilizing the information of the angle of view, the system 100 may adjust disinfection time or the radiation intensity so as to ensure disinfection quality.

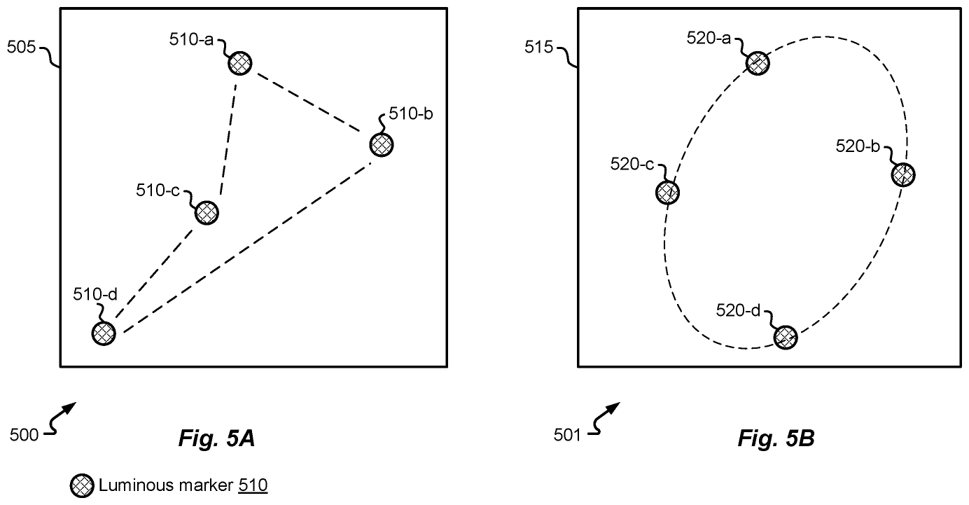

FIG. 5A illustrates an example configuration 500 a sensor tag 505 that supports a UV disinfection system with sensors and feedback in accordance with aspects of the present disclosure. FIG. 5B further illustrates an example configuration 501 of a sensor tag 515 that supports a UV disinfection system with sensors and feedback in accordance with aspects of the present disclosure. The sensor tag 505, markers 510, sensor tag 515, and markers 520 may include aspects of like elements described with reference to FIGS. 1 through 4.

The example configurations 500 and 501 support marker positioning that avoids symmetry for unique angular location calculations. In an example, the sensor tag 505 may include markers 510 (e.g., marker 510-a through marker 510-d) positioned asymmetric to one another with respect to a two-dimensional plane. The markers 510 as illustrated, for example, form a four-point object without an axis of symmetry in the two-dimensional plane.

In another example, the sensor tag 515 may include markers 520 (e.g., marker 520-a through marker 520-d) positioned on an elliptical shape in a two-dimensional plane. The markers 520, as illustrated, are positioned on the elliptical shape such that there is no axis of symmetry with respect to the markers 520 in the two-dimensional plane.

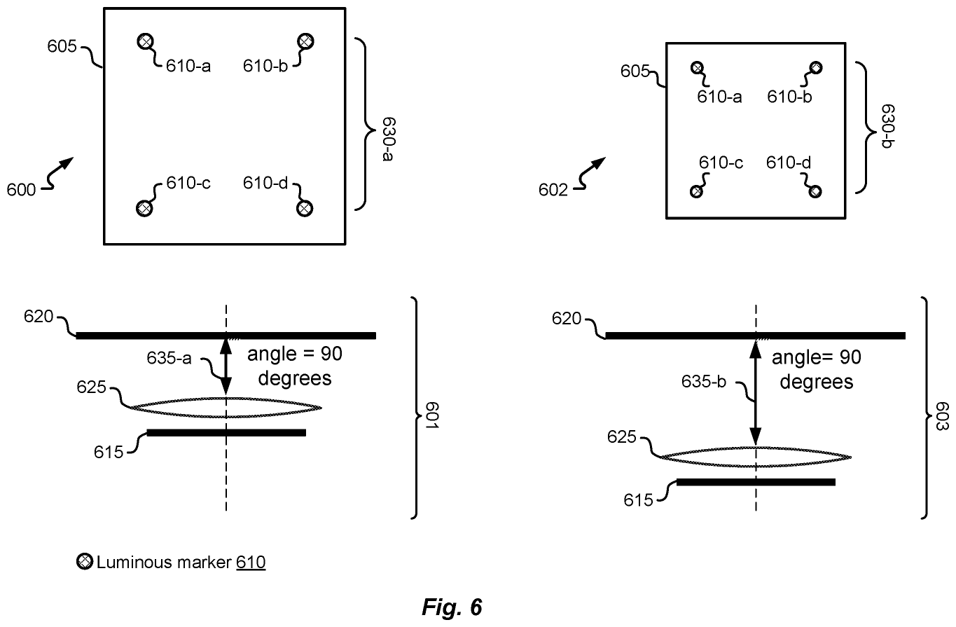

FIG. 6 illustrates example images 600 and 601 of a sensor tag 605 that supports a UV disinfection system with sensors and feedback in accordance with aspects of the present disclosure.

The example images 600 and 602 illustrate, for example, images captured by an image sensor 625. Referring to FIG. 6, the image sensor 625 is between an image sensor plane 615 (of the image sensor 625) and a tag plane 620 of the sensor tag 605. The sensor tag 605 may include markers 610 (e.g., marker 610-a through marker 610-d). The sensor tag 605, markers 610, and image sensor 625 may include aspects of like elements described with reference to FIGS. 1 through 5.

Image 600 illustrates an example of a captured image according to an example angular relationship 601, in which the image sensor 625 is a distance 630-a from the tag plane 620 of the sensor tag 605. Image 602 illustrates an example of a captured image according to an example angular relationship 603, in which the image sensor 625 is a distance 630-b from the tag plane 620.

In the example of FIG. 6, the image 600 is relatively larger than the image 602. That is, the same four-point object (e.g., defined by markers 610) of the sensor tag 605 is larger in the image 600 than in the image 602. In some aspects, distances (e.g., distance 630-a) between the markers 610 in the image 600 may be greater than distances (e.g., distance 630-b) between the markers 610 in the image 602.

Based on the size of the four-point object in the image 600, the image sensor 625 (e.g., or the system 100, communication device 105, server 110, or central controller described with reference to FIG. 1) may estimate a distance 635-a between the image sensor 625 and the tag plane 620. In another example, based on the size of the four-point object in the image 602, the image sensor 625 may estimate a distance 635-b between the image sensor 625 and the tag plane 620. The image sensor 625 (or the system 100) may determine, for example, that the distance 635-a (e.g., corresponding to the image 600 that is relatively larger), for example, is greater than the distance 635-b (e.g., corresponding to the image 602 that is relatively smaller). The estimated distance 635-b or 635a may be an input analyzed by the system 100 to control the disinfecting time and the radiation intensity so as to sufficiently disinfect the target area.

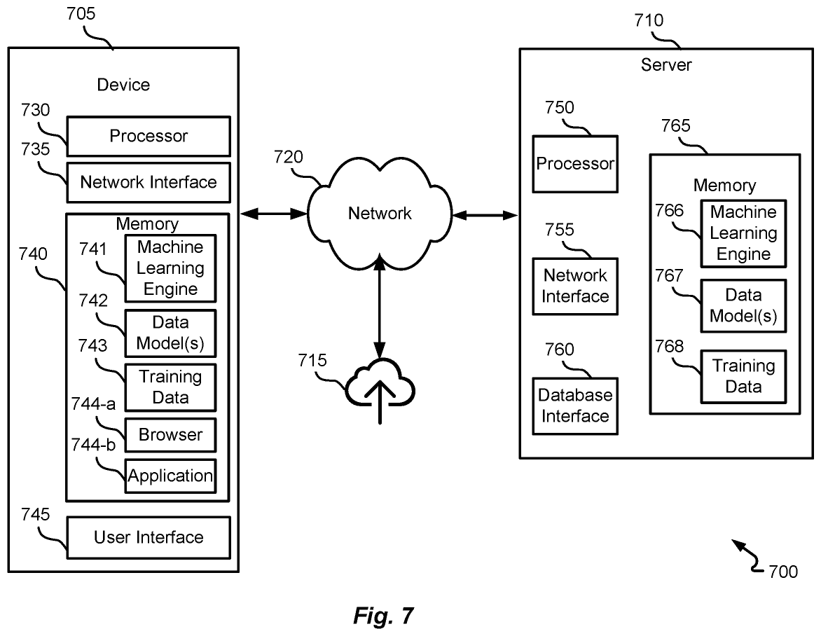

FIG. 7 illustrates an example of a system 700 that supports detecting and alerting a user when sending a message to an incorrect recipient or sending inappropriate content to a recipient in accordance with aspects of the present disclosure. In some examples, the system 700 may be implemented by aspects of the system 100 described with reference to FIG. 1. The system 700 may include a communication device 705, a server 710, a database 715, and a communication network 720. The communication devices 705, the server 710, the database 715, and the communications network 720 may be implemented, for example, like elements described herein.

In various aspects, settings of the any of the communication device 705, the server 110, database 715, and the network 720 may be configured and modified by any user and/or administrator of the system 700. Settings may include thresholds or parameters described herein, as well as settings related to how data is managed. Settings may be configured to be personalized for one or more communication devices 705, users of the communication devices 705, and/or other groups of entities, and may be referred to herein as profile settings, user settings, or organization settings. In some aspects, rules and settings may be used in addition to, or instead of, parameters or thresholds described herein. In some examples, the rules and/or settings may be personalized by a user and/or administrator for any variable, threshold, user (user profile), communication device 705, entity, or groups thereof.

A communication device 705 may include a processor 730, a network interface 735, a memory 740, and a user interface 745. In some examples, components of the communication device 705 (e.g., processor 730, network interface 735, memory 740, user interface 745) may communicate over a system bus (e.g., control busses, address busses, data busses) included in the communication device 705. In some cases, the communication device 705 may be referred to as a computing resource.

In some cases, the communication device 705 may transmit or receive packets to one or more other devices (e.g., another communication device 705, the server 710, the database 715) via the communication network 720, using the network interface 735. The network interface 735 may include, for example, any combination of network interface cards (NICs), network ports, associated drivers, or the like. Communications between components (e.g., processor 730, memory 740) of the communication device 705 and one or more other devices (e.g., another communication device 705, the database 715) connected to the communication network 720 may, for example, flow through the network interface 735.

The processor 730 may correspond to one or many computer processing devices. For example, the processor 730 may include a silicon chip, such as a FPGA, an ASIC, any other type of IC chip, a collection of IC chips, or the like. In some aspects, the processors may include a microprocessor, CPU, a GPU, or plurality of microprocessors configured to execute the instructions sets stored in a corresponding memory (e.g., memory 740 of the communication device 705). For example, upon executing the instruction sets stored in memory 740, the processor 730 may enable or perform one or more functions of the communication device 705.

The processor 730 may utilize data stored in the memory 740 as a neural network (also referred to herein as a machine learning network). The neural network may include a machine learning architecture. In some aspects, the neural network may be or include an artificial neural network (ANN). In some other aspects, the neural network may be or include any machine learning network such as, for example, a deep learning network, a convolutional neural network, or the like. Some elements stored in memory 740 may be described as or referred to as instructions or instruction sets, and some functions of the communication device 705 may be implemented using machine learning techniques.

The memory 740 may include one or multiple computer memory devices. The memory 740 may include, for example, Random Access Memory (RAM) devices, Read Only Memory (ROM) devices, flash memory devices, magnetic disk storage media, optical storage media, solid-state storage devices, core memory, buffer memory devices, combinations thereof, and the like. The memory 740, in some examples, may correspond to a computer-readable storage media. In some aspects, the memory 740 may be internal or external to the communication device 705.

The memory 740 may be configured to store instruction sets, neural networks, and other data structures (e.g., depicted herein) in addition to temporarily storing data for the processor 730 to execute various types of routines or functions. For example, the memory 740 may be configured to store program instructions (instruction sets) that are executable by the processor 730 and provide functionality of machine learning engine 741 described herein. The memory 740 may also be configured to store data or information that is useable or capable of being called by the instructions stored in memory 740. One example of data that may be stored in memory 740 for use by components thereof is a data model(s) 742 (also referred to herein as a neural network model) and/or training data 743 (also referred to herein as a training data and feedback).

The machine learning engine 741 may include a single or multiple engines. The communication device 705 (e.g., the machine learning engine 741) may utilize one or more data models 742 for recognizing and processing information obtained from other communication devices 705, the server 710, sensor tags (e.g., sensor tags 140 described with reference to FIG. 1), and the database 715. In some aspects, the communication device 705 (e.g., the machine learning engine 741) may update one or more data models 742 based on learned information included in the training data 743. In some aspects, the machine learning engine 741 and the data models 742 may support forward learning based on the training data 743. The machine learning engine 741 may have access to and use one or more data models 742. For example, the data model(s) 742 may be built and updated by the machine learning engine 741 based on the training data 743. The data model(s) 742 may be provided in any number of formats or forms. Non-limiting examples of the data model(s) 742 include Decision Trees, Support Vector Machines (SVMs), Nearest Neighbor, and/or Bayesian classifiers.

In some examples, the training data 743 may include aggregated intensity information with respect to one or more temporal periods and/or one or more disinfection sessions. In some other examples, the training data 743 may include parameters and/or configurations of a radiation source (e.g., location, orientation, emission power, emission duration, etc.). In some cases, the training data 743 may include parameters and/or configurations of an image sensor (e.g., location, orientation, focus settings, etc.) as described herein. In some aspects, the training data 743 may include parameters and/or configurations associated with established RF communications (e.g., established RF communications links, beamforming parameters, etc.) between the communication device 705 and a sensor tag.

The machine learning engine 741 may be configured to analyze aggregated intensity information, parameters, and/or configurations that are historical or in real-time. The machine learning engine 741 may be configured to receive or access information from the communication device 705, the server 710, and/or the database 715. The machine learning engine 741 may build any number of profiles (e.g., disinfection profiles associated with a physical environment, configuration profiles associated with a radiation source, configuration profiles associated with an image sensor, etc.) using automatic processing, using artificial intelligence and/or using input from one or more users associated with the communication device 705. The machine learning engine 741 may use automatic processing, artificial intelligence, and/or inputs from one or more users of the communication devices 705 to determine, manage, and/or combine information relevant to a configuration profile.

The machine learning engine 741 may determine configuration profile information based on a user's interactions with information. The machine learning engine 741 may update (e.g., continuously, periodically) configuration profiles based on new information that is relevant. The machine learning engine 741 may receive new information from any communication device 705, the server 710, the database 715, a sensor tag, etc. Profile information may be organized and classified in various manners. In some aspects, the organization and classification of configuration profile information may be determined by automatic processing, by artificial intelligence and/or by one or more users of the communication devices 705.

The machine learning engine 741 may create, select, and execute appropriate processing decisions. Example processing decisions may include analysis of aggregated intensity information, configuration of an image sensor, configuration of a radiation source, and configuration of a disinfection session. Processing decisions may be handled automatically by the machine learning engine 305, with or without human input.

The machine learning engine 741 may store, in the memory 740 (e.g., in a database included in the memory 740), historical information (e.g., aggregated intensity information, configurations, etc.). Data within the database of the memory 740 may be updated, revised, edited, or deleted by the machine learning engine 741. In some aspects, the machine learning engine 741 may support continuous, periodic, and/or batch fetching of data (e.g., from sensor tags, image sensors, radiation sources, a central controller, communication devices 705, etc.) and data aggregation.

The communication device 705 may render a presentation (e.g., visually, audibly, using haptic feedback, etc.) of an application 744 (e.g., a browser application 744-a, an application 744-b). The application 744-b may be an application associated with executing, controlling, and/or monitoring a disinfection session described herein. For example, the application 744-b may enable control of the communication device 705, an image sensor (e.g., image sensor 130), a radiation source (e.g., radiation source 125), or a transport instrument described herein.

In an example, the communication device 705 may render the presentation via the user interface 745. The user interface 745 may include, for example, a display (e.g., a touchscreen display), an audio output device (e.g., a speaker, a headphone connector), or any combination thereof. In some aspects, the applications 744 may be stored on the memory 740. In some cases, the applications 744 may include cloud-based applications or server-based applications (e.g., supported and/or hosted by the database 715 or the server 710). Settings of the user interface 745 may be partially or entirely customizable and may be managed by one or more users, by automatic processing, and/or by artificial intelligence.

In an example, any of the applications 744 (e.g., browser application 744-a, application 744-b) may be configured to receive data in an electronic format and present content of data via the user interface 745. For example, the applications 744 may receive data from another communication device 705, the server 710, an image sensor, a radiation source, or a sensor tag via the communications network 720, and the communication device 705 may display the content via the user interface 745.

The database 715 may include a relational database, a centralized database, a distributed database, an operational database, a hierarchical database, a network database, an object-oriented database, a graph database, a NoSQL (non-relational) database, etc. In some aspects, the database 715 may store and provide access to, for example, any of the stored data described herein.

The server 710 may include a processor 750, a network interface 755, database interface instructions 760, and a memory 765. In some examples, components of the server 710 (e.g., processor 750, network interface 755, database interface 760, memory 765) may communicate over a system bus (e.g., control busses, address busses, data busses) included in the server 710. The processor 750, network interface 755, and memory 765 of the server 710 may include examples of aspects of the processor 730, network interface 735, and memory 740 of the communication device 705 described herein.

For example, the processor 750 may be configured to execute instruction sets stored in memory 765, upon which the processor 750 may enable or perform one or more functions of the server 710. In some aspects, the processor 750 may utilize data stored in the memory 765 as a neural network. In some examples, the server 710 may transmit or receive packets to one or more other devices (e.g., a communication device 705, the database 715, another server 710) via the communication network 720, using the network interface 755. Communications between components (e.g., processor 750, memory 765) of the server 710 and one or more other devices (e.g., a communication device 705, the database 715, an image sensor, a radiation source, a sensor tag) connected to the communication network 720 may, for example, flow through the network interface 755.

In some examples, the database interface instructions 760 (also referred to herein as database interface 760), when executed by the processor 750, may enable the server 710 to send data to and receive data from the database 715. For example, the database interface instructions 760, when executed by the processor 750, may enable the server 710 to generate database queries, provide one or more interfaces for system administrators to define database queries, transmit database queries to one or more databases (e.g., database 715), receive responses to database queries, access data associated with the database queries, and format responses received from the databases for processing by other components of the server 710.

The memory 765 may be configured to store instruction sets, neural networks, and other data structures (e.g., depicted herein) in addition to temporarily storing data for the processor 750 to execute various types of routines or functions. For example, the memory 765 may be configured to store program instructions (instruction sets) that are executable by the processor 750 and provide functionality of the machine learning engine 766 described herein. One example of data that may be stored in memory 765 for use by components thereof is a data model(s) 767 (also referred to herein as a neural network model) and/or training data 768. The data model(s) 767 and the training data 768 may include examples of aspects of the data model(s) 742 and the training data 743 described with reference to the communication device 705. For example, the server 710 (e.g., the machine learning engine 766) may utilize one or more data models 767 for recognizing and processing information obtained from communication devices 705, another server 710, the database 715, an image sensor, a radiation source, and/or a sensor tag. In some aspects, the server 710 (e.g., the machine learning engine 766) may update one or more data models 767 based on learned information included in the training data 768.

In some aspects, components of the machine learning engine 766 may be provided in a separate machine learning engine in communication with the server 710.

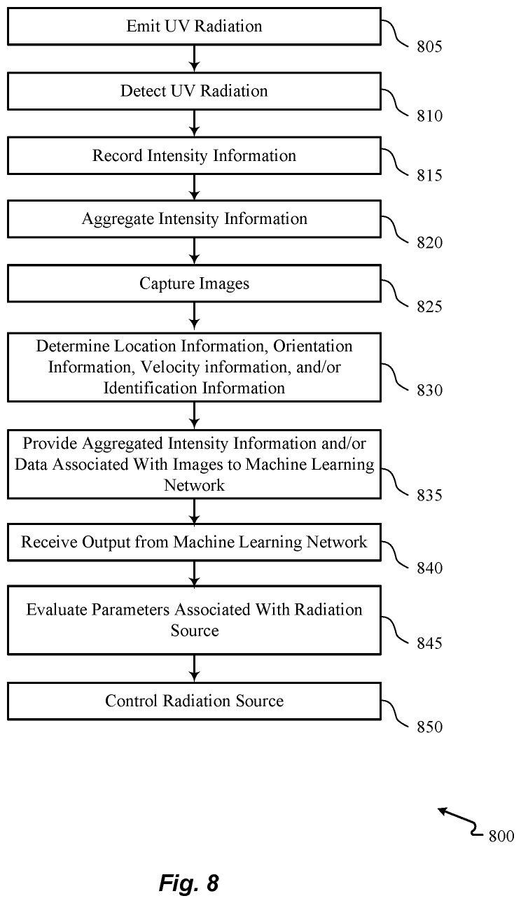

FIG. 8 illustrates an example of a process flow 800 that supports a UV disinfection system with sensors and feedback in accordance with aspects of the present disclosure. In some examples, process flow 800 may implement aspects of system 100 or system 700. Further, process flow 800 may be implemented by a system 100 or components included therein as described with reference to FIGS. 1 through 7.

In the following description of the process flow 800, the operations may be performed in a different order than the order shown, or the operations may be performed in different orders or at different times. Certain operations may also be left out of the process flow 800, or other operations may be added to the process flow 800. It is to be understood that any device (e.g., a communication device 105, a server 110, a central controller, components of the system 100, etc.) may perform the operations shown.

At 805, the system 100 may emit UV radiation via a radiation source (e.g., radiation source 125).

At 810, the system 100 (e.g., sensor tag 140) may detect the UV radiation.

At 815, the system 100 (e.g., sensor tag 140) may record intensity information associated with the UV radiation. In some aspects, recording the intensity information may include recording temporal information associated with the intensity information, the temporal information including a timestamp value.

At 820, the system 100 (e.g., central controller) may aggregate the intensity information based on the recording of the intensity information. In some aspects, aggregating the intensity information may be based on the temporal information.

In some aspects, recording the intensity information at 815 may include: recording first intensity information associated with the UV radiation, first temporal information associated with the first intensity information, or both; recording second intensity information associated with the UV radiation, second temporal information associated with the second intensity information, or both. In some aspects, aggregating the intensity information at 820 may include aggregating the first intensity information and the second intensity information based on comparison of the first temporal information and the second information.

At 825, the system 100 (e.g., image sensor 130) may capture one or more images of a physical environment including one or more sensor devices (e.g., one or more sensor tags 140).

In some examples, at 830, based on the one or more images, the system 100 (e.g., central controller) may determine at least one of: location information associated with the one or more sensor devices; orientation information associated with the one or more sensor devices; velocity information associated with the one or more sensor devices; and identification information associated with the one or more sensor devices.

In some examples, at 835, the system 100 may provide at least a portion of the aggregated intensity information, at least a portion of data associated with the one or more images, or both to a machine learning network.

At 840, the system 100 may receive an output from the machine learning network in response to the machine learning network processing at least the portion of the aggregated intensity information, at least the portion of the data, or both.

In some aspects, the output from the machine learning network may include at least one of: a predicted radiation coverage corresponding to a target area and a temporal period; probability information corresponding to the predicted radiation coverage; and confidence information associated with the probability information. In some aspects, the output from the machine learning network may include at least one of: predicted location information associated with the one or more sensor devices and a predicted temporal period; predicted orientation information associated with the one or more sensor devices and the predicted temporal period; and predicted velocity information associated with the one or more sensor devices and the predicted temporal period. In some aspects, the output from the machine learning network further may include at least one of: probability information corresponding to the predicted location information, the predicted orientation information, the predicted velocity information, or a combination thereof; and confidence information associated with the probability information.

At 845, the system 100 (e.g., central controller) may evaluate one or more parameters associated with the radiation source based on the aggregated intensity information. In an example, evaluating the one or more parameters associated with the radiation source may be based on capturing the one or more images. In an example, evaluating the one or more parameters associated with the radiation source may be based on the output from the machine learning network.

In some aspects, the one or more parameters associated with the radiation source may include at least one of: a degradation level associated with the emitted UV radiation; a degradation rate associated with the emitted UV radiation; a coverage area associated with the emitted UV radiation; and a quality level of a disinfection session.

At 850, the system 100 (e.g., central controller) may control, based on the output from the machine learning network, at least one of: a location of the radiation source; an emission direction of the radiation source; an emission power of the radiation source; and an emission duration of the radiation source.

In some aspects not illustrated in FIG. 8, the system 100 (e.g., central controller, communication device 105, RF transceiver 135) may generate a directed beam for communicating RF signals with the one or more sensor devices based on the location information, the orientation information, the velocity information, or a combination thereof. The system 100 may establish a communications link with the one or more sensor devices based on the directed beam.

In some aspects not illustrated in FIG. 8, the system 100 (e.g., central controller, communication device 105, RF transceiver 135) may receive an indicator from one or more sensor devices included in a physical environment. In some aspects, the indicator may be associated with intensity information recorded by the one or more sensor devices, a pathogen level detected by the one or more sensor devices, or both. The system 100 (e.g., central controller, communication device 105, server 110) may pause emission of the UV radiation by the radiation source or resume the emission based on the receiving the indicator. Alternatively, or additionally, the system 100 (e.g., central controller, communication device 105, server 110) may modify at least one of a position, a location, an orientation, and an emission direction of the radiation source based on the indicator.

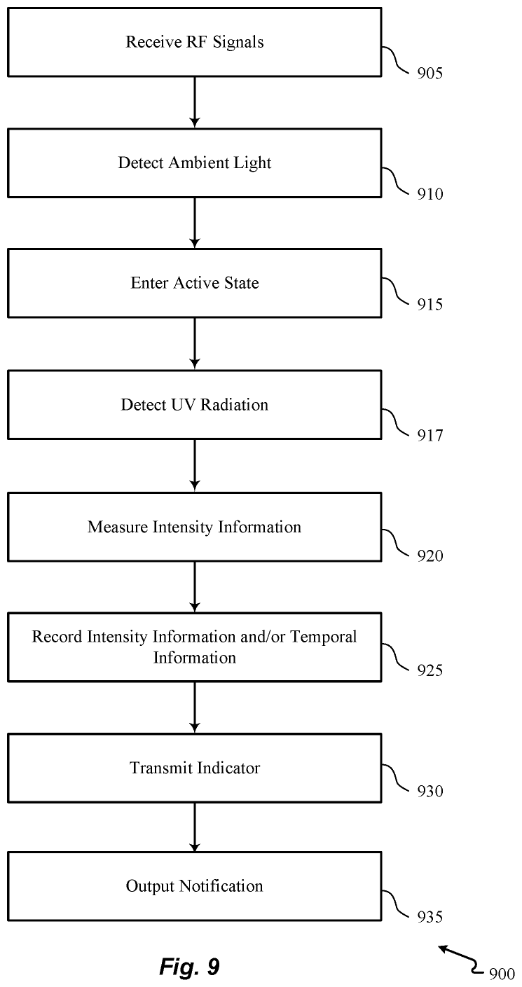

FIG. 9 illustrates an example of a process flow 900 that supports a UV disinfection system with sensors and feedback in accordance with aspects of the present disclosure. In some examples, process flow 900 may implement aspects of a sensor tag 140 (also referred to herein as a sensor device) described with reference to FIG. 1. Further, process flow 900 may be implemented by a sensor tag described with reference to any of FIGS. 2 through 7.

In the following description of the process flow 900, the operations may be performed in a different order than the order shown, or the operations may be performed in different orders or at different times. Certain operations may also be left out of the process flow 900, or other operations may be added to the process flow 900.

At 905, the sensor tag 140 may receive radio frequency (RF) signals during a temporal period. In some aspects, the RF signals may energize the sensor tag 140 (e.g., as part of a global wake-up described herein). In some aspects, the RF signals may include temporal reference data.

At 910, the sensor tag 140 may detect, at a light sensor of the device, light having a wavelength between 380 nm to 780 nm (e.g., ambient light).

At 915, the sensor tag 140 may enter an active state (e.g., an awake state) based on receiving the RF signals. Alternatively, or additionally, at 915, the sensor tag 140 may enter the active state based on an amount of ambient light detected by the light sensor satisfying a threshold.

At 917, the sensor tag 140 may detect UV radiation (also referred to herein as UV light) of a first frequency band. In an example, the first frequency band may be from 100 nm to 280 nm (e.g., UV-C band).

At 920, the sensor tag 140 may measure intensity information associated with UV radiation of the first frequency band.

At 925, the sensor tag 140 may record the intensity information, temporal information associated with measuring the intensity information, or both. In some aspects, the temporal information may include a timestamp value synchronized with the temporal reference data.

At 930, the sensor tag 140 may transmit an indicator based on at least one of: comparing the recorded intensity information and a first threshold; and comparing a pathogen level associated with a target area and a second threshold. In some aspects, the indicator may include a command associated with pausing or enabling emissions of the UV radiation (e.g., pausing emissions of UV radiation emissions by the radiation source 125).

At 935, the sensor tag 140 may output a notification based on a comparison of the recorded intensity information and a first threshold. In some aspects, the notification may include at least one of: a visual notification; an audible notification; and a haptic notification.

Any of the steps, functions, and operations discussed herein can be performed continuously and automatically.

The exemplary systems and methods of this disclosure have been described in relation to examples of a system 100 (e.g., communication device 105, radiation source 125, image sensor 130, RF transceiver 135, sensor tags 140). However, to avoid unnecessarily obscuring the present disclosure, the preceding description omits a number of known structures and devices. This omission is not to be construed as a limitation of the scope of the claimed disclosure. Specific details are set forth to provide an understanding of the present disclosure. It should, however, be appreciated that the present disclosure may be practiced in a variety of ways beyond the specific detail set forth herein.

Furthermore, while the exemplary embodiments illustrated herein show the various components of the system collocated, certain components of the system can be located remotely, at distant portions of a distributed network, such as a LAN and/or the Internet, or within a dedicated system. Thus, it should be appreciated, that the components of the system can be combined into one or more devices, such as a server, communication device, or collocated on a particular node of a distributed network, such as an analog and/or digital telecommunications network, a packet-switched network, or a circuit-switched network. It will be appreciated from the preceding description, and for reasons of computational efficiency, that the components of the system can be arranged at any location within a distributed network of components without affecting the operation of the system.

Furthermore, it should be appreciated that the various links connecting the elements can be wired or wireless links, or any combination thereof, or any other known or later developed element(s) that is capable of supplying and/or communicating data to and from the connected elements. These wired or wireless links can also be secure links and may be capable of communicating encrypted information. Transmission media used as links, for example, can be any suitable carrier for electrical signals, including coaxial cables, copper wire, and fiber optics, and may take the form of acoustic or light waves, such as those generated during radio-wave and infra-red data communications.

While the flowcharts have been discussed and illustrated in relation to a particular sequence of events, it should be appreciated that changes, additions, and omissions to this sequence can occur without materially affecting the operation of the disclosed embodiments, configuration, and aspects.

A number of variations and modifications of the disclosure can be used. It would be possible to provide for some features of the disclosure without providing others.

In yet another embodiment, the systems and methods of this disclosure can be implemented in conjunction with a special purpose computer, a programmed microprocessor or microcontroller and peripheral integrated circuit element(s), an ASIC or other integrated circuit, a digital signal processor, a hard-wired electronic or logic circuit such as discrete element circuit, a programmable logic device or gate array such as PLD, PLA, FPGA, PAL, special purpose computer, any comparable means, or the like. In general, any device(s) or means capable of implementing the methodology illustrated herein can be used to implement the various aspects of this disclosure. Exemplary hardware that can be used for the present disclosure includes computers, handheld devices, telephones (e.g., cellular, Internet enabled, digital, analog, hybrids, and others), and other hardware known in the art. Some of these devices include processors (e.g., a single or multiple microprocessors), memory, nonvolatile storage, input devices, and output devices. Furthermore, alternative software implementations including, but not limited to, distributed processing or component/object distributed processing, parallel processing, or virtual machine processing can also be constructed to implement the methods described herein.

In yet another embodiment, the disclosed methods may be readily implemented in conjunction with software using object or object-oriented software development environments that provide portable source code that can be used on a variety of computer or workstation platforms. Alternatively, the disclosed system may be implemented partially or fully in hardware using standard logic circuits or VLSI design. Whether software or hardware is used to implement the systems in accordance with this disclosure is dependent on the speed and/or efficiency requirements of the system, the particular function, and the particular software or hardware systems or microprocessor or microcomputer systems being utilized.

In yet another embodiment, the disclosed methods may be partially implemented in software that can be stored on a storage medium, executed on programmed general-purpose computer with the cooperation of a controller and memory, a special purpose computer, a microprocessor, or the like. In these instances, the systems and methods of this disclosure can be implemented as a program embedded on a personal computer such as an applet, JAVA® or CGI script, as a resource residing on a server or computer workstation, as a routine embedded in a dedicated measurement system, system component, or the like. The system can also be implemented by physically incorporating the system and/or method into a software and/or hardware system.

Although the present disclosure describes components and functions implemented in the embodiments with reference to particular standards and protocols, the disclosure is not limited to such standards and protocols. Other similar standards and protocols not mentioned herein are in existence and are considered to be included in the present disclosure. Moreover, the standards and protocols mentioned herein and other similar standards and protocols not mentioned herein are periodically superseded by faster or more effective equivalents having essentially the same functions. Such replacement standards and protocols having the same functions are considered equivalents included in the present disclosure.

The present disclosure, in various embodiments, configurations, and aspects, includes components, methods, processes, systems and/or apparatus substantially as depicted and described herein, including various embodiments, subcombinations, and subsets thereof. Those of skill in the art will understand how to make and use the systems and methods disclosed herein after understanding the present disclosure. The present disclosure, in various embodiments, configurations, and aspects, includes providing devices and processes in the absence of items not depicted and/or described herein or in various embodiments, configurations, or aspects hereof, including in the absence of such items as may have been used in previous devices or processes, e.g., for improving performance, achieving ease, and/or reducing cost of implementation.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more embodiments, configurations, or aspects for the purpose of streamlining the disclosure. The features of the embodiments, configurations, or aspects of the disclosure may be combined in alternate embodiments, configurations, or aspects other than those discussed above. This method of disclosure is not to be interpreted as reflecting an intention that the claimed disclosure requires more features than are expressly recited in each claim. Rather, as the following claims reflect, inventive aspects lie in less than all features of a single foregoing disclosed embodiment, configuration, or aspect. Thus, the following claims are hereby incorporated into this Detailed Description, with each claim standing on its own as a separate preferred embodiment of the disclosure.

Moreover, though the description of the disclosure has included description of one or more embodiments, configurations, or aspects and certain variations and modifications, other variations, combinations, and modifications are within the scope of the disclosure, e.g., as may be within the skill and knowledge of those in the art, after understanding the present disclosure. It is intended to obtain rights, which include alternative embodiments, configurations, or aspects to the extent permitted, including alternate, interchangeable and/or equivalent structures, functions, ranges, or steps to those claimed, whether or not such alternate, interchangeable and/or equivalent structures, functions, ranges, or steps are disclosed herein, and without intending to publicly dedicate any patentable subject matter.

The phrases “at least one,” “one or more,” “or,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” “A, B, and/or C,” and “A, B, or C” means A alone, B alone, C alone, A and B together, A and C together, B and C together, or A, B and C together.

The term “a” or “an” entity refers to one or more of that entity. As such, the terms “a” (or “an”), “one or more,” and “at least one” can be used interchangeably herein. It is also to be noted that the terms “comprising,” “including,” and “having” can be used interchangeably.

The term “automatic” and variations thereof, as used herein, refers to any process or operation, which is typically continuous or semi-continuous, done without material human input when the process or operation is performed. However, a process or operation can be automatic, even though performance of the process or operation uses material or immaterial human input, if the input is received before performance of the process or operation. Human input is deemed to be material if such input influences how the process or operation will be performed. Human input that consents to the performance of the process or operation is not deemed to be “material.”

Aspects of the present disclosure may take the form of an embodiment that is entirely hardware, an embodiment that is entirely software (including firmware, resident software, micro-code, etc.) or an embodiment combining software and hardware aspects that may all generally be referred to herein as a “circuit,” “module,” or “system.” Any combination of one or more computer-readable medium(s) may be utilized. The computer-readable medium may be a computer-readable signal medium or a computer-readable storage medium.

A computer-readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer-readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer-readable storage medium may be any tangible medium that can contain or store a program for use by or in connection with an instruction execution system, apparatus, or device.

A computer-readable signal medium may include a propagated data signal with computer-readable program code embodied therein, for example, in baseband or as part of a carrier wave. Such a propagated signal may take any of a variety of forms, including, but not limited to, electro-magnetic, optical, or any suitable combination thereof. A computer-readable signal medium may be any computer-readable medium that is not a computer-readable storage medium and that can communicate, propagate, or transport a program for use by or in connection with an instruction execution system, apparatus, or device. Program code embodied on a computer-readable medium may be transmitted using any appropriate medium, including, but not limited to, wireless, wireline, optical fiber cable, RF, etc., or any suitable combination of the foregoing.

The terms “determine,” “calculate,” “compute,” and variations thereof, as used herein, are used interchangeably and include any type of methodology, process, mathematical operation or technique.

What is claimed is:

1. A system comprising:
a radiation source configured to emit ultraviolet (UV) radiation;
one or more sensor devices, the one or more sensor devices comprising a sensor configured to detect the UV radiation, wherein the one or more sensor devices are configured to record intensity information associated with the UV radiation; and
a central controller configured to:
aggregate the intensity information recorded by the one or more sensor devices; and
evaluate one or more parameters associated with the radiation source based at least in part on the aggregated intensity information.

2. The system of claim 1, further comprising:
an image sensor configured to capture one or more images of a physical environment comprising the one or more sensor devices,
wherein evaluating the one or more parameters associated with the radiation source is based at least in part on capturing the one or more images.

3. The system of claim 2, wherein:
the one or more sensor devices comprise a location sensor; and
the central controller is further configured to determine, based at least in part on the one or more images, the location sensor, or both at least one of:
location information associated with the one or more sensor devices;
orientation information associated with the one or more sensor devices;
velocity information associated with the one or more sensor devices; and
identification information associated with the one or more sensor devices.

4. The system of claim 2, wherein:
the radiation source is configured to generate a directed beam for communicating RF signals with the one or more sensor devices based at least in part on the location information, the orientation information, the velocity information, or a combination thereof; and
the central controller is further configured to establish a communications link with the one or more sensor devices based at least in part on the directed beam.

5. The system of claim 2, further comprising:
a machine learning network;
wherein the central controller is further configured to:
provide at least a portion of the aggregated intensity information, at least a portion of data associated with the one or more images, or both to a machine learning network; and
receive an output from the machine learning network in response to the machine learning network processing at least the portion of the aggregated intensity information, at least the portion of the data, or both,
wherein evaluating the one or more parameters associated with the radiation source is based at least in part on the output from the machine learning network.

6. The system of claim 5, wherein the output from the machine learning network comprises at least one of:
a predicted radiation coverage corresponding to a target area and a temporal period;
probability information corresponding to the predicted radiation coverage; and
confidence information associated with the probability information.

7. The system of claim 5, wherein the output from the machine learning network comprises at least one of:
predicted location information associated with the one or more sensor devices and a predicted temporal period;
predicted orientation information associated with the one or more sensor devices and the predicted temporal period; and
predicted velocity information associated with the one or more sensor devices and the predicted temporal period.

8. The system of claim 7, wherein the output from the machine learning network further comprises at least one of:
probability information corresponding to the predicted location information, the predicted orientation information, the predicted velocity information, or a combination thereof; and
confidence information associated with the probability information.

9. The system of claim 5, wherein, based at least in part on the output from the machine learning network, the central controller is further configured to control at least one of:
a location of the radiation source;
an emission direction of the radiation source;
an emission power of the radiation source; and
an emission duration of the radiation source.

10. The system of claim 1, wherein:
the one or more sensor devices are configured to record temporal information associated with the intensity information recorded by the sensor, the temporal information comprising a timestamp value; and
the central controller is further configured to aggregate the intensity information based at least in part on the temporal information recorded by the one or more sensor devices.

11. The system of claim 10, wherein the one or more sensor devices comprise:
a first sensor device configured to record first intensity information associated with the UV radiation, first temporal information associated with the first intensity information, or both;
a second sensor device configured to record second intensity information associated with the UV radiation, second temporal information associated with the second intensity information, or both;
wherein the central controller is configured to aggregate the first intensity information and the second intensity information based at least in part on comparison of the first temporal information and the second information.

12. The system of claim 1, wherein the one or more parameters associated with the radiation source comprise at least one of:
a degradation level associated with the emitted UV radiation;
a degradation rate associated with the emitted UV radiation;
a coverage area associated with the emitted UV radiation; and
a quality level of a disinfection session.

13. The system of claim 2, wherein the one or more sensor devices further comprise:
a housing; and
a radiation sensitive material disposed on one or more portions of an external surface of the housing, wherein the image sensor is further configured to visually detect the one or more sensor devices based at least in part on the one or more images, the radiation sensitive material, or both.

14. The system of claim 13, wherein:
the radiation sensitive material is disposed on the external surface of the housing according to a first pattern, a second pattern, or both, wherein the first pattern includes a set of points formed of the radiation sensitive material, and the second pattern includes a one-dimensional pattern-based code formed of the radiation sensitive material, a multi-dimensional pattern-based code formed of the radiation sensitive material, or both; and
the central controller is configured to at least one of:
determine the location information, the orientation information, or both based at least in part on the set of points; and
determine the identification information based at least in part on the one-dimensional pattern-based code, the multi-dimensional pattern-based code, or both.

15. The system of claim 1, further comprising:
a server; and
a source device coupled to the radiation source,
wherein the central controller is comprised in a processor of the server or the source device.

16. The system of claim 1, further comprising:
a first transceiver device configured for radio frequency (RF) communications; and
a source device coupled to the first transceiver device,
wherein the source device is configured to transmit, via the first transceiver device, temporal reference data, wherein the one or more sensor devices are configured to synchronize with the temporal reference data.

17. The system of claim 1, wherein:
the radiation source comprises or is electrically coupled to a location sensor: and
the central controller is further configured to determine, based at least in part on the location sensor of the radiation source, at least one of:
location information associated with the radiation source;
orientation information associated with the radiation source; and
velocity information associated with the radiation source.

18. The system of claim 1, wherein:
the one or more sensor devices are configured to transmit an indicator based at least in part on at least one of:
a comparison of the recorded intensity information to a first threshold; and
a comparison of a pathogen level detected by the one or more sensor devices with respect to a target area to a second threshold.

19. A method comprising:
emitting ultraviolet (UV) radiation via a radiation source;
detecting the UV radiation;
recording intensity information associated with the UV radiation; and
aggregating the intensity information based at least in part on the recording; and
evaluating one or more parameters associated with the radiation source based at least in part on the aggregated intensity information.

20. The method of claim 19, further comprising:
capturing, by an image sensor, one or more images of a physical environment comprising one or more sensor devices,
wherein evaluating the one or more parameters associated with the radiation source is based at least in part on capturing the one or more images.
